(19) United States
(12) Patent Application Publication
(10) Pub. No.: US 2003/0199483 A1
(43) Pub. Date: Oct. 23, 2003

(54) Aldosterone receptor antagonist and alpha-adrenergic modulating agent combination therapy for prevention or treatment of pathogenic conditions

(76) Inventors: Ellen G. McMahon, Sunset Hills, MO (US); Amy Rudolph, St. Louis, MO (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/354,653

(22) Filed: Jan. 30, 2003

Related U.S. Application Data
(60) Provisional application No. 60/353,801, filed on Jan. 30, 2002.

(51) Int. Cl. A61K 31/56; A61K 31/496; A61K 31/4745; A61K 31/47

(52) U.S. Cl. 514/171; 514/283; 514/307; 514/355; 514/252.17

(57) ABSTRACT

A combination therapy comprising a therapeutically-effective amount of an aldosterone receptor antagonist and a therapeutically-effective amount of an alpha-adrenergic modulating agent is described for treatment of circulatory disorders, including cardiovascular disorders such as hypertension, congestive heart failure, cirrhosis and ascites. Preferred alpha-adrenergic modulating agents are those compounds having high potency and bioavailability. Preferred aldosterone receptor antagonists are 20-spiroxane steroidal compounds characterized by the presence of a 9α,11α-substituted epoxy moiety. A preferred combination therapy includes an alpha-1-adrenergic antagonist or an alpha-2-adrenergic agonist and the aldosterone receptor antagonist epoxymexrenone.

### FIELD OF THE INVENTION

Combinations of an aldosterone receptor antagonist and an alpha-adrenergic modulating agent are described for use in prevention or treatment of pathogenic conditions, including circulatory disorders encompassing cardiovascular diseases such as hypertension, heart failure (including congestive heart failure), cardiac hypertrophy, cirrhosis and ascites. Of particular interest are therapies using an epoxy-containing steroidal aldosterone receptor antagonist compound such as epoxymexrenone in combination with either an alpha-1-adrenergic antagonist compound or an alpha-2-adrenergic agonist compound.

### BACKGROUND OF THE INVENTION

Myocardial (or cardiac) failure, whether a consequence of a previous myocardial infarction, heart disease associated with hypertension, or primary cardiomyopathy, is a major health problem of worldwide proportions. The incidence of symptomatic heart failure has risen steadily over the past several decades.

In clinical terms, decompensated cardiac failure consists of a constellation of signs and symptoms that arises from congested organs and hypoperfused tissues to form the congestive heart failure (CHF) syndrome. Congestion is caused largely by increased venous pressure and by inadequate sodium (Na⁺) excretion, relative to dietary Na⁺ intake, and is importantly related to circulating levels of aldosterone (ALDO). An abnormal retention of Na⁺ occurs via tubular epithelial cells throughout the nephron, including the later portion of the distal tubule and cortical collecting ducts, where ALDO receptor sites are present.

ALDO is the body's most potent mineralocorticoid hormone. As connoted by the term mineralocorticoid, this steroid hormone has mineral-regulating activity. It promotes Na⁺ reabsorption not only in the kidney, but also from the lower gastrointestinal tract and salivary and sweat glands, each of which represents classic ALDO-responsive tissues. ALDO regulates Na and water resorption at the expense of potassium (K⁺) and magnesium (Mg²⁺) excretion.

ALDO can also provoke responses in nonepithelial cells. Elicited by a chronic elevation in plasma ALDO level that is inappropriate relative to dietary Na⁺ intake, these responses can have adverse consequences on the structure of the cardiovascular system. Hence, ALDO can contribute to the progressive nature of myocardial failure for multiple reasons.

Multiple factors regulate ALDO synthesis and metabolism, many of which are operative in the patient with myocardial failure. These include renin as well as non-renin-dependent factors (such as K⁺, ACTH) that promote ALDO synthesis. Hepatic blood flow, by regulating the clearance of circulating ALDO, helps determine its plasma concentration, an important factor in heart failure characterized by reduction in cardiac output and hepatic blood flow.

The renin-angiotensin-aldosterone system (RAAS) is one of the hormonal mechanisms involved in regulating pressure/volume homeostasis and also in the development of hypertension. Activation of the renin-angiotensin-aldosterone system begins with renin secretion from the juxtaglomerular cells in the kidney and culminates in the formation of angiotensin II, the primary active species of this system. This octapeptide, angiotensin II, is a potent vasoconstrictor and also produces other physiological effects such as stimulating aldosterone secretion, promoting sodium and fluid retention, inhibiting renin secretion, increasing sympathetic nervous system activity, stimulating vasopressin secretion, causing positive cardiac inotropic effect and modulating other hormonal systems.

In addition to aldosterone, alpha-adrenergic receptors play an important role in circulatory disorders. For example, in vascular tissue muscle cell contraction occurs when cells are stimulated by catecholamines binding to alpha-1-adrenergic receptors. In peripheral tissues this can lead to systemic hypertension. Alpha-1-adrenergic antagonists block this effect and cause vasodilation, reduced blood pressure (anti-hypertensive effect) and a reduction in the force required to pump blood by the heart. In addition, alpha-1-adrenergic receptors can mediate pathogenic hypertrophy in various tissues (including the heart and prostate gland—benign prostatic hypertrophy) and apoptosis, both of which can be inhibited with an alpha-1-adrenergic antagonist. Stimulation of alpha-2-adrenergic receptors, presumably located in the cardiovascular control centers of the CNS, can cause a reduction in sympathetic nervous system activity. As a result, administration of alpha-2-adrenergic receptor agonists produces a decrease in blood pressure.

Many aldosterone receptor blocking drugs are known. For example, spironolactone is a drug which acts at the mineralocorticoid receptor level by competitively inhibiting aldosterone binding. This steroidal compound has been used for blocking aldosterone-dependent sodium transport in the distal tubule of the kidney in order to reduce edema and to treat essential hypertension and primary hyperaldosteronism [F. Mantero et al, Clin. Sci. Mol. Med., 45 (Suppl 1), 219s-224s (1973)]. Spironolactone is also used commonly in the treatment of other hyperaldosterone-related diseases such as liver cirrhosis and congestive heart failure [F. J. Saunders et al, Aldactone; Spironolactone: A Comprehensive Review, Searle, New York (1978)]. Progressively-increasing doses of spironolactone from 1 mg to 400 mg per day [i.e., 1 mg/day, 5 mg/day, 20 mg/day] were administered to a spironolactone-intolerant patient to treat cirrhosis-related ascites [P. A. Greenberger et al, N. Eng. Reg. Allergy Proc., 7(4), 343-345 (July-August, 1986)]. It has been recognized that development of myocardial fibrosis is sensitive to circulating levels of both Angiotensin II and aldosterone, and that the aldosterone antagonist spironolactone prevents myocardial fibrosis in animal models, thereby linking aldosterone to excessive collagen deposition [D. Klug et al, Am. J. Cardiol., 71 (3), 46A-54A (1993)]. Spironolactone has been shown to prevent fibrosis in animal models irrespective of the development of left ventricular hypertrophy and the presence of hypertension [C. G. Brilla et al, J. Mol. Cell. Cardiol., 25(5), 563-575 (1993)]. Spironolactone at a dosage ranging from 25 mg to 100 mg daily is used to treat diuretic-induced hypokalemia, when orally-administered potassium supplements or other potassium-sparing regimens are considered inappropriate [Physicians' Desk Reference, 46th Edn., p. 2153, Medical Economics Company Inc., Montvale, N.J. (1992)].

Another series of steroidal-type aldosterone receptor antagonists is exemplified by epoxy-containing spironolactone derivatives. For example, U.S. Pat. No. 4,559,332 issued to Grob et al describes 9a,11a-epoxy-containing spironolactone derivatives as aldosterone antagonists useful as diuretics. These 9a,11a-epoxy steroids have been evaluated for endocrine effects in comparison to spironolactone [M. de Gasparo et al, J. Pharm. Exp. Ther., 240(2), 650-656 (1987)].

The combination of an aldosterone receptor antagonist with clonidine, guanfacine, guanabenz, prazosin, terazosin, doxazosin or labetalol has been disclosed in U.S. patent application Ser. No. 2002/0,132,001.

### SUMMARY OF THE INVENTION

A combination therapy comprising a therapeutically-effective amount of an aldosterone receptor antagonist and a therapeutically-effective amount of an alpha-adrenergic modulating agent is useful to prevent or treat pathogenic conditions, including circulatory disorders encompassing cardiovascular diseases such as hypertension, congestive heart failure, cirrhosis and ascites.

The phrase “alpha-adrenergic modulating agent” is intended to embrace one or more compounds or agents having the ability to

1) act as an “alpha-1-adrenergic antagonist” by interacting with and blocking alpha-1-adrenergic receptors located in various tissues, wherein the alpha-1-adrenergic receptor activity is associated with initiating or mediating one or more biological functions or events which are pathogenic, or

2) act as an “alpha-2-adrenegic agonist” by interacting with and activating alpha-2-adrenergic receptors located in various tissues, wherein the alpha-2-adrenergic receptor activity is associated with inhibiting or blocking one or more biological functions or events which are pathogenic.

The phrase “aldosterone antagonist” denotes a compound capable of binding to an aldosterone receptor, as a competitive inhibitor of the action of aldosterone itself at the receptor site, so as to modulate the receptor-mediated activity of aldosterone.

The phrase “epoxy-steroidal aldosterone receptor antagonist” is intended to embrace one or more agents or compounds characterized by a steroid-type nucleus and having an epoxy moiety attached to the nucleus and which agent or compound binds to the aldosterone receptor, as a competitive inhibitor of the action of aldosterone itself at the receptor site, so as to modulate the receptor-mediated activity of aldosterone.

The phrase “combination therapy”, in defining use of an alpha-adrenergic modulating agent and an aldosterone receptor antagonist, is intended to embrace administration of each antagonist in a sequential manner in a regimen that will provide beneficial effects of the drug combination, and is intended to embrace co-administration of the antagonist agents in a substantially simultaneous manner, such as in a single capsule having a fixed ratio of active ingredients or in multiple, separate capsules for each antagonist agent.

The phrase “therapeutically-effective” is intended to qualify the amount of each active agent (for example, aldosterone receptor antagonist or alpha-adrenergic modulating agent or the combination of these agents), for use in combination therapy, that will achieve the goal of preventing or ameliorating a pathogenic condition. This goal may include improvement in disorder severity, or the frequency of incidence occurrence requiring medical attention or hospitalization. Example of this goal would include reducing hypertension with improvement in cardiac sufficiency, preventing the progression of congestive heart failure, and reducing mortality or morbidity.

The term “treatment” or “treating” includes the administration, to a person in need, of an amount of an aldosterone antagonist and alpha-adrenergic modulating agent combination which will prevent or ameliorate development of a pathological condition.

The term “prevent”, “prevention” or “preventing” includes either preventing the onset of clinically evident pathogenic disorders altogether or preventing the onset of a preclinically evident stage of cardiovascular disorder in individuals. This includes prophylactic treatment of those at risk of developing a circulatory disorder.

The term “subject” for purposes of treatment includes any human or animal subject who is susceptible to or suffering from a pathogenic disorder, and preferably is a human subject. The subject, for example, may be at risk due to diet, exposure to bacterial or viral infection, having common markers present, being genetically predisposed to cardiovascular disorders, and the like.

The amount of aldosterone receptor antagonist blocker that is administered and the dosage regimen for the methods of this invention depend on a variety of factors, including the age, weight, sex and medical condition of the subject, the severity of the pathogenic effect, the route and frequency of administration, and the particular aldosterone blocker employed, and thus may vary widely. A daily dose administered to a subject of about 0.001 to 30 mg/kg body weight, preferably between about 0.005 and about 20 mg/kg body weight, more preferably between about 0.01 and about 15 mg/kg body weight, still more preferably between about 0.05 and about 10 mg/kg body weight, and most preferably between about 0.01 to 5 mg/kg body weight, may be appropriate.

The daily dose of aldosterone antagonist administered to a human subject typically will range from about 0.1 mg to about 2000 mg. In one embodiment of the present invention, the daily dose range is from about 0.1 mg to about 400 mg. In another embodiment of the present invention, the daily dose range is from about 1 mg to about 200 mg. In a further embodiment of the present invention, the daily dose range is from about 1 mg to about 100 mg. In another embodiment of the present invention, the daily dose range is from about 10 mg to about 100 mg. In a further embodiment of the present invention, the daily dose range is from about 25 mg to about 100 mg. In another embodiment of the present invention, the daily dose is selected from the group consisting of 5 mg, 10 mg, 12.5 mg, 25 mg, 50 mg, 75 mg, and 100 mg. In a further embodiment of the present invention, the daily dose is selected from the group consisting of 25 mg, 50 mg, and 100 mg. A daily dose of aldosterone blocker that produces no substantial diuretic and/or anti-hypertensive effect in a subject is specifically embraced by the present method. The daily dose can be administered in one to four doses per day.

Dosing of the aldosterone blocker can be determined and adjusted based on measurement of parameters that would be known to one skilled in the art. Non-limiting examples of such parameters would include blood pressure or appropriate surrogate markers (such as natriuretic peptides, endothelins, and other surrogate markers). Blood pressure and/or surrogate marker levels after administration of the aldosterone blocker can be compared against the corresponding baseline levels prior to administration of the aldosterone blocker to determine efficacy of the present method and titrated as needed. Non-limiting examples of surrogate markers useful in the method are surrogate markers for renal and cardiovascular disease.

For a combination of an alpha-adrenergic modulating agent and an ALDO antagonist agent, the agents would be used in combination in a weight ratio range from about one-to-0.5 to about one-to-twenty of the alpha-adrenergic modulating agent to the aldosterone receptor antagonist agent. A preferred range of these two agents (alpha-adrenergic modulating agent-to-ALDO antagonist) would be from about one-to-one to about one-to-fifteen, while a more preferred range would be from about one-to-one to about one-to-five, depending ultimately on the selection of the alpha-adrenergic modulating agent and ALDO antagonist.

### DETAILED DESCRIPTION OF THE INVENTION

The aldosterone antagonists used in the methods of the present invention generally are spirolactone-type steroidal compounds. The term “spirolactone-type”, is intended to characterize a structure comprising a lactone moiety attached to a steroid nucleus, typically at the steroid “D” ring, through a spiro bond configuration. A subclass of spirolactone-type aldosterone antagonist compounds consists of epoxy-steroidal aldosterone antagonist compounds such as eplerenone. Another subclass of spirolactone-type antagonist compounds consists of non-epoxy-steroidal aldosterone antagonist compounds such as spironolactone.

The epoxy-steroidal aldosterone antagonist compounds used in the method of the present invention generally have a steroidal nucleus substituted with an epoxy-type moiety. The term “epoxy-type” moiety is intended to embrace any moiety characterized in having an oxygen atom as a bridge between two carbon atoms, examples of which include the following moieties:

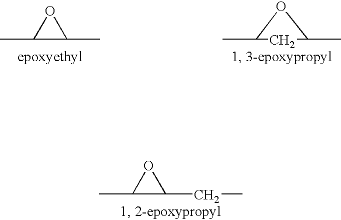

The term “steroidal”, as used in the phrase “epoxy-steroidal”, denotes a nucleus provided by a cyclopenteno-phenanthrene moiety, having the conventional “A”, “B”, “C” and “D” rings. The epoxy-type moiety may be attached to the cyclopentenophenanthrene nucleus at any attachable or substitutable positions, that is, fused to one of the rings of the steroidal nucleus or the moiety may be substituted on a ring member of the ring system. The phrase “epoxy-steroidal” is intended to embrace a steroidal nucleus having one or a plurality of epoxy-type moieties attached thereto.

Epoxy-steroidal aldosterone antagonists suitable for use in the present methods include a family of compounds having an epoxy moiety fused to the “C” ring of the steroidal nucleus. Especially preferred are 20-spiroxane compounds characterized by the presence of a 9α,11α-substituted epoxy moiety. Compounds 1 through 11, Table 1 below, are illustrative 9α,11α-epoxy-steroidal compounds that may be used in the present methods. These epoxy steroids may be prepared by procedures described in Grob et al., U.S. Pat. No. 4,559,332. Additional processes for the preparation of 9,11-epoxy steroidal compounds and their salts are disclosed in Ng et al., WO97/21720 and Ng et al., WO98/25948.

TABLE 1
Aldosterone Receptor Antagonist

| Com- | |
|---|---|
| pound # | Structure and Name |
| 1 | 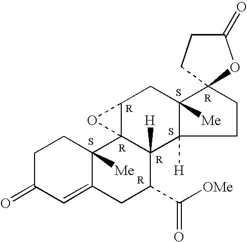 |
| | Pregn-4-ene-7,21-dicarboxylic acid, 9,11-epoxy- |
| | 17-hydroxy-3-oxo-, γ-lactone, methyl ester, (7α, 11α, 17β) - |
| 2 | 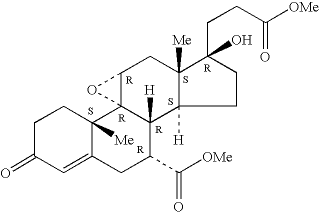 |
| | Pregn-4-ene-7,21-dicarboxylic acid, 9,11-epoxy- |
| | 17-hydroxy-3-oxo-, dimethyl ester, (7α, 11α, 17β) - |
| 3 | 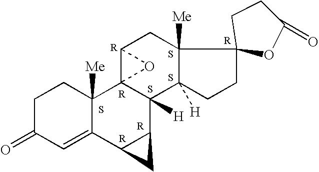 |
| | 3′H-cyclopropa[6,7]pregna-4,6-diene-21-carboxylic |
| | acid, 9,11-epoxy-6,7-dihydro-17-hydroxy-3-oxo-, |
| | γ-lactone, (6β, 7β, 11α, 17β) - |
| 4 | 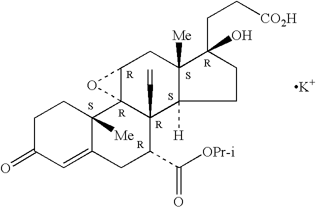 |
| | Pregn-4-ene-7,21-dicarboxylic acid, 9,11-epoxy-17- |
| | hydroxy-3-oxo-, 7-(1-methylethyl) ester, |
| | monopotassium salt, (7α, 11α, 17β) - |
| 5 | 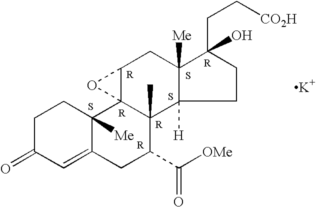 |
| | Pregn-4-ene-7,21-dicarboxylic acid, 9,11-epoxy-17- |
| | hydroxy-3-oxo-, 7-methylethyl) ester, monopotassium |
| | salt, (7α, 11α, 17β) - |
| 6 | 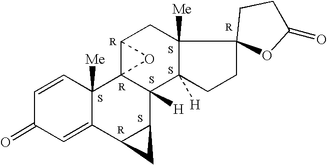 |
| | 3′H-cyclopropa[6,7]pregna-1,4,6-triene-21-carboxylic |
| | acid, 9,11-epoxy-6,7-dihydro-17-hydroxy-3-oxo-, |
| | γ-lactone (6β, 7β, 11α) - |
| 7 | 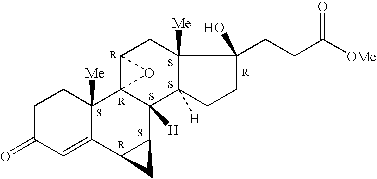 |
| | 3′H-cyclopropa[6,7]pregna-4,6-diene-21-carboxylic |
| | acid, 9,11-epoxy-6,7-dihydro-17-hydroxy-3-oxo-, |
| | methyl ester, (6β, 7β, 11α, 17β) - |
| 8 | 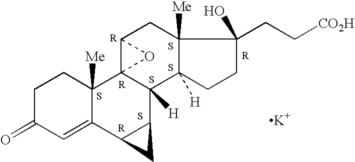 |
| | 3′H-cyclopropa[6,7]pregna-4,6-diene-21-carboxylic |
| | acid, 9,11-epoxy-6,7-dihydro-17-hydroxy-3-oxo-, |
| | monopotassium salt, (6β, 7β, 11α, 17β) - |
| 9 | 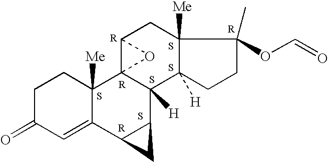 |
| | 3′H-cyclopropa[6,7]pregna-1,4,6-triene-21-carboxylic |
| | acid, 9,11-epoxy-6,7-dihydro-17-hydroxy-3-oxo-, γ- |
| | lactone (6β, 7β, 11α, 17β) - |
| 10 | 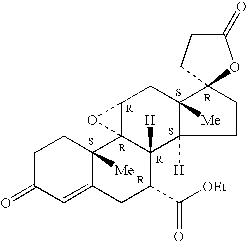 |
| | Pregn-4-ene-7,21-dicarboxylic acid, 9,11-epoxy- |
| | 17-hydroxy-3-oxo-, γ-lactone, ethyl ester, (7α, 11α, 17β) - |
| 11 | 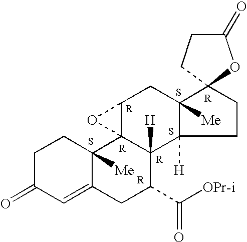 |
| | Pregn-4-ene-7,21-dicarboxylic acid, 9,11-epoxy- |
| | 17-hydroxy-3-oxo-, γ-lactone, 1-methylethyl |
| | ester (7α, 11α, 17β) - |

Of particular interest is the compound eplerenone (also known as: epoxymexrenone and CGP 30 083) which is compound 1 as shown above. The chemical name for eplerenone is pregn-4-ene-7,21-dicarboxylic acid, 9,11-epoxy-17-hydroxy-3-oxo, γ-lactone, methyl ester, (7α,11α,17α)-. This chemical name corresponds to the CAS 10 registry name for eplerenone (the CAS registry number for eplerenone is 107724-20-9). U.S. Pat. No. 4,559,332 identifies eplerenone by the alternative name of 9α,11α-epoxy-7α-methoxycarbonyl-20-spirox-4-ene-3,21-dione. Such “spiroxane” nomenclature is further described, for example, at column 2, line 16 through column 4, line 48 of U.S. Pat. No. 4,559,332. Eplerenone is an aldosterone receptor antagonist and has a higher specificity for aldosterone receptors than does, for example, spironolactone. Selection of eplerenone as the aldosterone antagonist in the present method would be beneficial to reduce certain side-effects such as gynecomastia that occur with use of aldosterone antagonists having less specificity.

Non-epoxy-steroidal aldosterone antagonists suitable for use in the present methods include a family of spirolactone-type compounds defined by Formula I:

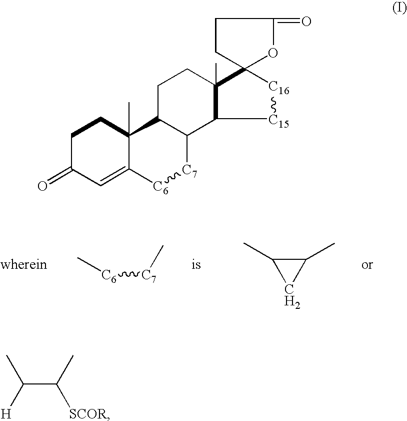

wherein R is lower alkyl of up to 5 carbon atoms, and

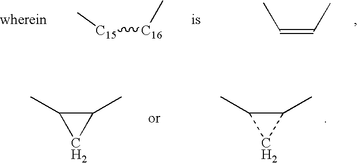

Lower alkyl residues include branched and unbranched groups, preferably methyl, ethyl and n-propyl.

Specific compounds of interest within Formula I are the following:

7α-acetylthio-3-oxo-4,15-androstadiene-[17(β-1′)-spiro-5′]perhydrofuran-2′-one;

3-oxo-7α-propionylthio-4,15-androstadiene-[17((β-1′)-spiro-5′]perhydrofuran-2′-one;

6β,7β-methylene-3-oxo-4,15-androstadiene-[17((β-1′)-spiro-5′]perhydrofuran-2′-one;

15α,16═-methylene-3-oxo-4,7α-propionylthio-4-androstene-[17(β-1′)-spiro-5′]perhydrofuran-2′-one;

6β,7β,15α,16α-dimethylene-3-oxo-4-androstene[17(β-1′)-spiro-5′]-perhydrofuran-2′-one;

7α-acetylthio-15β,16β-Methylene-3-oxo-4-androstene-[17(β-1′)-spiro-5′]perhydrofuran-2′-one;

15β,16β-methylene-3-oxo-7β-propionylthio-4-androstene-[17(β-1′)-spiro-5′]perhydrofuran-2′-one; and

6β,7β,15β,16β-dimethylene-3-oxo-4-androstene-[17(β-1′)-spiro-5′]perhydrofuran-2′-one.

Methods to make compounds of Formula I are described in U.S. Pat. No. 4,129,564 to Wiechart et al. issued on Dec. 12, 1978.

Another family of non-epoxy-steroidal compounds of interest is defined by Formula II:

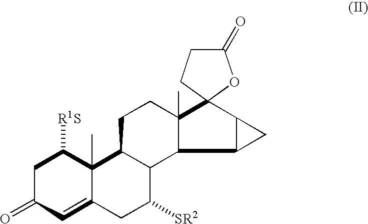

wherein R¹ is C₁₋₃-alkyl or C₁₋₃acyl and R² is H or C₁₋₃-alkyl.

Specific compounds of interest within Formula II are the following:

1α-acetylthio-15β,16β-methylene-7α-methylthio-3-oxo-17α-pregn-4-ene-21,17-carbolactone; and

15β,16β-methylene-1α,7α-dimethylthio-3-oxo-17α-pregn-4-ene-21,17-carbolactone.

Methods to make the compounds of Formula II are described in U.S. Pat. No. 4,789,668 to Nickisch et al. which issued Dec. 5, 1988.

Yet another family of non-epoxy-steroidal compounds of interest is defined by a structure of Formula III:

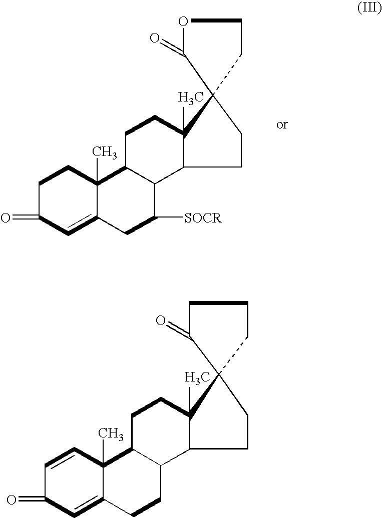

wherein R is lower alkyl, with preferred lower alkyl groups being methyl, ethyl, propyl and butyl. Specific compounds of interest include:

3β,21-dihydroxy-17α-pregna-5,15-diene-17-carboxylic acid γ-lactone;

3β,21-dihydroxy-17α-pregna-5,15-diene-17-carboxylic acid γ-lactone 3-acetate;

3β,21-dihydroxy-17α-pregn-5-ene-17-carboxylic acid γ-lactone;

3β,21-dihydroxy-17α-pregn-5-ene-17-carboxylic acid γ-lactone 3-acetate;

21-hydroxy-3-oxo-17α-pregn-4-ene-17-carboxylic acid γ-lactone;

21-hydroxy-3-oxo-17α-pregna-4,6-diene-17-carboxylic acid γ-lactone;

21-hydroxy-3-oxo-17α-pregna-1,4-diene-17-carboxylic acid γ-lactone;

7α-acylthio-21-hydroxy-3-oxo-17α-pregn-4-ene-17-carboxylic acid γlactone; and

7α-acetylthio-21-hydroxy-3-oxo-17α-pregn-4-ene-17-carboxylic acid γ-lactone.

Methods to make the compounds of Formula III are described in U.S. Pat. No. 3,257,390 to Patchett which issued Jun. 21, 1966.

Still another family of non-epoxy-steroidal compounds of interest is represented by Formula IV:

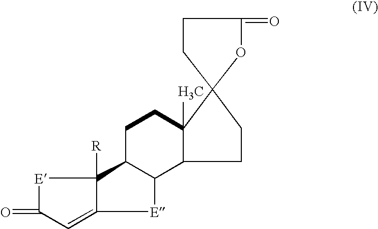

wherein E′ is selected from the group consisting of ethylene, vinylene and (lower alkanoyl)thioethylene radicals, E″ is selected from the group consisting of ethylene, vinylene, (lower alkanoyl)thioethylene and (lower alkanoyl)thiopropylene radicals; R is a methyl radical except when E′ and E″ are ethylene and (lower alkanoyl) thioethylene radicals, respectively, in which case R is selected from the group consisting of hydrogen and methyl radicals; and the selection of E′ and E″ is such that at least one (lower alkanoyl)thio radical is present.

A preferred family of non-epoxy-steroidal compounds within Formula IV is represented by Formula V:

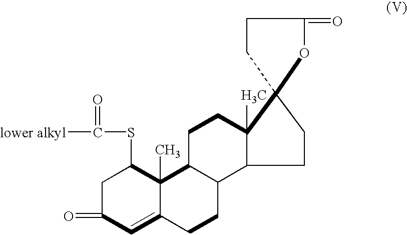

A more preferred compound of Formula V is 1-acetylthio-17α-(2-carboxyethyl)-17β-hydroxy-androst-4-en-3-one lactone.

Another preferred family of non-epoxy-steroidal compounds within Formula IV is represented by Formula VI:

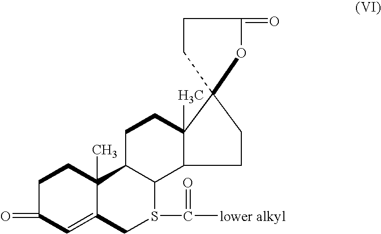

More preferred compounds within Formula VI include the following:

7α-acetylthio-17α-(2-carboxyethyl)-17β-hydroxy-androst-4-en-3-one lactone;

7β-acetylthio-17α(2-carboxyethyl)-17β-hydroxy-androst-4-en-3-one lactone;

1α,7α-diacetylthio-17α-(2-carboxyethyl)-17β-hydroxy-androsta-4,6-dien-3-one lactone;

7α-acetylthio-17α-(2-carboxyethyl)-17β-hydroxy-androsta-1,4-dien-3-one lactone;

7α-acetylthio-17α-(2-carboxyethyl)-17β-hydroxy-19-norandrost-4-en-3-one lactone; and

7α-acetylthio-17α-(2-carboxyethyl)-17β-hydroxy-6α-methylandrost-4-en-3-one lactone;

In Formulae IV-VI, the term “alkyl” is intended to embrace linear and branched alkyl radicals containing one to about eight carbons. The term “(lower alkanoyl)thio” embraces radicals of the formula lower

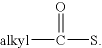

Of particular interest is the compound spironolactone having the following structure and formal name:

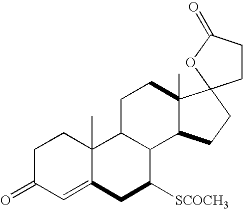

“spironolactone”: 17-hydroxy-7α-mercapto-3-oxo-17α-pregn-4-ene-21-carboxylic acid γ-lactone acetate.

Methods to make compounds of Formulae IV-VI are described in U.S. Pat. No. 3,013,012 to Cella et al. which issued Dec. 12, 1961. Spironolactone is sold by G. D. Searle & Co., Skokie, Ill., under the trademark “ALDACTONE”, in tablet dosage form at doses of 25 mg, 50 mg and 100 mg per tablet.

Another family of steroidal aldosterone antagonists is exemplified by drospirenone, [6R-6alpha,7alpha,8beta, 9alpha,10beta,13beta,14alpha,15alpha,16alpha, 17beta)]-1,3′,4′,6,7,8,9,10,11,12,13,14,15,16,20,21-hexadecahydro-10,13-dimethylspiro[17H-dicyclopropa[6,7:15,16]cyclopenta[a]phenanthrene-17,2′(5′H)-furan]-3,5′(2H)-dione, CAS registration number 67392-87-4. Methods to make and use drospirenone are described in patent GB 1550568 1979, priority DE 2652761 1976.

Tables 2 and 3, below, describe alpha-adrenergic modulating agents that may be used in the combination therapy. Each published document listed in Tables 2 and 3 describes important aspects of the associated alpha-adrenergic modulating agent, such as the chemical preparation or the biological properties of such compound. The content of each of these documents is incorporated herein by reference.

TABLE 2
Alpha-1-Adrenergic Antagonists

| Compound | Chemical | |
|---|---|---|
| Name | Name/CAS | Reference |
| amosulalol | 5-[1-hydroxy-2-[[2-(2- | U.S. Pat. No. 4,217,307 |
| | methoxyphenoxy)ethyl] | |
| | amino]ethyl]-2-methyl | |
| | benzenesulfonamide/ | |
| | 85320-68-9 | |
| arotinolol | (+,−)-5-[2-[[3-[(1,1- | U.S. Pat. No. 3,932,400 |
| | dimethylethyl)amino]-2- | |
| | hydroxypropyl]thio]-4- | |
| | thiazolyl]-2- | |
| | thiophenecarboxamide/ | |
| | 68377-92-4 | |
| dapiprazole | 5,6,7,8-tetrahydro-3-[2- | U.S. Pat. No. 4,252,721 |
| | [4-(2-methylphenyl)-1- | |
| | piperazinyl]ethyl]-1,2,4- | |
| | triazolo [4,3-a]pyridine/ | |
| | 72822-12-9 | |
| doxazosin | 1-(4-amino-6,7- | U.S. Pat. No. 4,188,390 |
| | dimethoxy-2- | |
| | quinazolinyl)-4-[(2,3- | |
| | dihydro-1,4- | |
| | benzodioxin-2-yl)c | |
| | arbonyl]piperizine/ | |
| | 74191-85-8 | |
| fenspirlde | | U.S. Pat. No. 3,399,192 |
| indoramin | N-[1-[2-(1H-indol-3- | U.S. Pat. No. 3,527,761 |
| | yl)ethyl]-4- | |
| | piperidinyl]benzamide/ | |
| | 26844-12-2 | |
| labetalol | 5-[1-hydroxy-2-1(1- | U.S. Pat. No. 4,012,444 |
| | methyl-3-phenylpropyl) | |
| | amino] | |
| | ethyl]salicylamide | |
| | monohydrochloride / | |
| | 36894-69-6 | |
| naftopidil | 4-(2-methoxyphenyl)- | U.S. Patent No. 3,997,666 |
| | alpha-[(1- | |
| | naphthalenyloxy)methyl]- | |
| | 1-piperazineethanol / | |
| | 57149-07-2 | |
| nicergoline | 27848-84-6 | U.S. Pat. No. 3,228,943 |
| prazosin | 19216-56-9 | U.S. Pat. No. 3,511,836 |
| tamsulosin | R-(−)-5-[2-[[2-(2- | U.S. Pat. No. 4,703,063 |
| | ethoxyphenoxy)ethyl] | |
| | amino]propyl]-2- | |
| | methoxybenzenesulfon- | |
| | amide / 106133-20-4 | |
| tolazoline | | U.S. Pat. No. 2,161,938 |
| trimazosin | | U.S. Pat. No. 3,669,968 |
| yohimbine | | Raymond-Hamet, J. Pharm. |
| | | Chim., 19, 209 (1934) |
| phenoxy- | N-(2 Chloroethyl)-N- | |
| benzamine | (1-methyl-2- | |
| | phenoxyethyl)benzylamine | |
| | hydrochloride / | |
| phentolamine | 3-[[(4,5-dihydro-1H- | U.S. Pat. No. 2,503,059 |
| | imidazol-2-yl)methyl](4- | |
| | methylphenyl)amino] | |
| | phenol / 50-60-2 | |
| terazosin | 1-(4-amino-6,7- | GB 1517403 |
| | dimethoxy-2- | |
| | quinazolinyl)-4- | |
| | [(tetrahydro-2- | |
| | furanyl)carbonyl] | |
| | piperazine / 63590-64-7 | |
| bunazosin | 1-(4-amino-6,7- | GB 1398455 |
| | dimethoxy-2- | |
| | quinazolinyl)-4- | |
| | butyrylhexahydro-1H- | |
| | 1,4-diazepine / | |
| | 80755-51-7 | |
| urapidil | 6-[[3-[4-(2- | GB 1309324 |
| | methoxyphenyl)-1- | |
| | piperazinyl]propyl]amino]- | |
| | 1,3-dimethyl- | |
| | 2,4(1H,3H)- | |
| | pyrimidinedione / | |
| | 34661-75-1 | |
| alfuzosin | N-[3-[(4-amino-6,7- | GB 2013679 |
| | dimethoxy-2- | |
| | quinazolinyl)methylamino] | |
| | propyl]tetrahydro-2- | |
| | furancarboxamide / | |
| | 81403-80-7 | |
| ketanserin | 3-[2-[4-(4-fluorobenzyl)- | EP 13612 |
| | 1-piperidinyl]ethyl]- | |
| | 2,4(1H,3H)- | |
| | quinazolinedione / | |
| | 74050-98-9 | |
| monatepil | (+,−)-N-(6,11- | EP 191867 |
| | dihydrodibenzo[b,e] | |
| | thiepin-11-yl)-4-(4- | |
| | fluorophenyl)-1- | |
| | piperazinebutanamide / | |
| | 132019-54-6 | |
| SUN 9221 | 2-(4-chlorophenyl)-1,4- | U.S. Pat. No. 4268516 |
| | dihydro- | |
| | [1]benzothiopyrano[4,3- | |
| | c]pyrazol-3(2H)-one 5- | |
| | oxide / 77606-94-1 | |
| S-2150 | (+)-cis-3-acetoxy-8- | EP 541263 B 1995 |
| | chloro-2,3-dihydro-2-(4- | |
| | methoxyphenyl)-5-[3-[4- | |
| | (2-methoxyphenyl)-1- | |
| | piperazinyl]propyl]-1,5- | |
| | benzothiazepin-4(5H)- | |
| | one citrate / | |
| | 149882-23-5 (2S-cis); | |
| | 149882-25-7 (2R-cis) | |

TABLE 3
Alpha-2-Adrenergic Agonists

| | Chemical | |
|---|---|---|
| Compound Name | Name/CAS | Reference |
| clonidine | 2-(2,6- | |
| | dichlorophenylamino)- | |
| | 2-imidazoline | |
| | hydrochloride / | |
| apraclonidine | 2,6-dichloro-N′-(4,5- | EP 81924 |
| | dihydro-1H- | |
| | imidazol-2-yl)-1,4- | |
| | benzenediamine / | |
| | 66711-21-5 | |
| guanfacine | N-amidino-2-(2,6- | |
| | dichlorophenyl) | |
| | acetamide | |
| | hydrochloride | |
| guanabenz | | |
| guanfacil | | |
| rilmenidine | | |
| moxonidine | 4-chloro-N-(4,5- | GB 2039275 |
| | dihydro-1H-imidazol-2- | |
| | yl)-6-methoxy-2- | |
| | methyl-5- | |
| | pyrimidinamine / | |
| | 75438-57-2 | |

In one embodiment, the alpha-adrenergic modulating agent is selected from the group consisting of dapiprazole, doxazosin, labetalol, prazosin, tamsulosin, tolazoline, phenoxybenzamine, phentolamine, terazosin, apraclonidine, clonidine, guanfacine, guanabenz, amosulalol, arotinolol, fenspirlde, indoramin, naftopidil, nicergoline, trimazosin, yohimbine, bunazosin, urapidil, alfuzosin, ketanserin, monatepil, SUN 9221, S-2150, guanfacil, rilmenidine, and moxonidine.

In another embodiment, the alpha-adrenergic modulating agent is selected from the group consisting of dapiprazole, doxazosin, labetalol, prazosin, tamsulosin, tolazoline, phenoxybenzamine, phentolamine, terazosin, apraclonidine, clonidine, guanfacine, and guanabenz.

In a further embodiment, the alpha-adrenergic modulating agent is selected from the group consisting of amosulalol, arotinolol, fenspirlde, indoramin, naftopidil, nicergoline, trimazosin, yohimbine, bunazosin, urapidil, alfuzosin, ketanserin, monatepil, SUN 9221, S-2150, guanfacil, rilmenidine, and moxonidine.

In one embodiment, the alpha-adrenergic modulating agent is an alpha-1-adrenergic antagonist selected from the group consisting of amosulalol, arotinolol, dapiprazole, doxazosin, fenspirlde, indoramin, labetalol, naftopidil, nicergoline, prazosin, tamsulosin, tolazoline, trimazosin, yohimbine, phenoxybenzamine, phentolamine, terazosin, bunazosin, urapidil, alfuzosin, ketanserin, monatepil, SUN 9221, and S-2150.

In another embodiment, the alpha-adrenergic modulating agent is an alpha-1-adrenergic antagonist selected from the group consisting of amosulalol, arotinolol, dapiprazole, doxazosin, fenspirlde, indoramin, labetalol, and naftopidil.

In another embodiment, the alpha-adrenergic modulating agent is an alpha-1-adrenergic antagonist selected from the group consisting of nicergoline, prazosin, tamsulosin, tolazoline, trimazosin, yohimbine, phenoxybenzamine, and phentolamine.

In a further embodiment, the alpha-adrenergic modulating agent is an alpha-1-adrenergic antagonist selected from the group consisting of terazosin, bunazosin, urapidil, alfuzosin, ketanserin, monatepil, SUN 9221, and S-2150.

In one embodiment, the alpha-adrenergic modulating agent is an alpha-2-adrenergic agonist selected from the group consisting of clonidine, apraclonidine, guanfacine, guanabenz, guanfacil, rilmenidine, and moxonidine.

In another embodiment, the alpha-adrenergic modulating agent is an alpha-2-adrenergic agonist selected from the group consisting of clonidine, apraclonidine, and guanfacil.

In a further embodiment, the alpha-adrenergic modulating agent is an alpha-2-adrenergic agonist selected from the group consisting of guanfacine, guanabenz, rilmenidine, and moxonidine.

In one embodiment, the alpha-adrenergic modulating agent is selected from the group consisting of dapiprazole, tamsulosin, tolazoline, phenoxybenzamine, phentolamine, apraclonidine, amosulalol, arotinolol, fenspirlde, indoramin, naftopidil, nicergoline, trimazosin, yohimbine, bunazosin, urapidil, alfuzosin, ketanserin, monatepil, SUN 9221, S-2150, guanfacil, rilmenidine, and moxonidine.

In a further embodiment, the alpha-adrenergic modulating agent is selected from the group consisting of dapiprazole, tamsulosin, tolazoline, phenoxybenzamine, phentolamine, and apraclonidine.

In one embodiment, the alpha-adrenergic modulating agent is an alpha-1-adrenergic antagonist selected from the group consisting of amosulalol, arotinolol, dapiprazole, fenspirlde, indoramin, naftopidil, nicergoline, tamsulosin, tolazoline, trimazosin, yohimbine, phenoxybenzamine, phentolamine, bunazosin, urapidil, alfuzosin, ketanserin, monatepil, SUN 9221, and S-2150.

In another embodiment, the alpha-adrenergic modulating agent is an alpha-1-adrenergic antagonist selected from the group consisting of amosulalol, arotinolol, dapiprazole, fenspirlde, indoramin, and naftopidil.

In another embodiment, the alpha-adrenergic modulating agent is an alpha-1-adrenergic antagonist selected from the group consisting of nicergoline, tamsulosin, tolazoline, trimazosin, yohimbine, phenoxybenzamine, and phentolamine.

In a further embodiment, the alpha-adrenergic modulating agent is an alpha-1-adrenergic antagonist selected from the group consisting of bunazosin, urapidil, alfuzosin, ketanserin, monatepil, SUN 9221, and S-2150.

In one embodiment, the alpha-adrenergic modulating agent is an alpha-2-adrenergic agonist selected from the group consisting of apraclonidine, guanfacil, rilmenidine, and moxonidine.

In another embodiment, the alpha-adrenergic modulating agent is an alpha-2-adrenergic agonist selected from the group consisting of apraclonidine, and guanfacil.

In a further embodiment, the alpha-adrenergic modulating agent is an alpha-2-adrenergic agonist selected from the group consisting of rilmenidine, and moxonidine.

The combination therapy of the invention would be useful in treating a variety of pathogenic conditions, including benign prostatic hypertrophy, glaucoma and other ophthalmological diseases, erectile dysfunction, pheochromocytoma and a number of circulatory disorders, including cardiovascular disorders, such as hypertension, heart failure (including congestive heart failure), myocardial fibrosis and cardiac hypertrophy. The combination therapy would also be useful with adjunctive therapies. For example, the combination therapy may be used in combination with other drugs, such as a non-aldosterone-receptor-antagonist-type diuretic, to aid in treatment of hypertension. The combination therapy would also be useful with adjunctive therapies comprising three or more compounds selected from one or more alpha-adrenergic modulating agents in combination with one or more aldosterone receptor antagonists.

Previous studies have shown that alpha-adrenergic modulating agents can be used successfully to treat hypertension. In heart failure this effect reduces the pressure load on the pumping heart, improving circulatory efficiency.

Aldosterone levels are elevated in heart failure patients. In addition to the pathogenic effects of aldosterone already noted, aldosterone also has been found to inhibit the uptake and removal of catecholamines in the myocardium. This results in higher cardiac adrenergic stimulation, which furthers the development of heart failure. The use of an aldosterone antagonist to treat such a patient, increases the rate of catecholamine uptake, reducing the cardiac concentration.

Accordingly, coadministration of an aldosterone antagonist, such as but not limited to eplerenone, ameliorates pathogenic consequences of catecholamines, thus enhancing the beta-adrenergic antagonist through coaction of the aldosterone receptor antagonist and the alpha-adrenergic modulating agent.

Definitions

The term “hydrido” denotes a single hydrogen atom (H). This hydrido group may be attached, for example, to an oxygen atom to form a hydroxyl group; or, as another example, one hydrido group may be attached to a carbon atom to form a

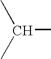

group; or, as another example, two hydrido atoms may be attached to a carbon atom to form a —CH₂— group. Where the term “alkyl” is used, either alone or within other terms such as “haloalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. The term “cycloalkyl” embraces cyclic radicals having three to about ten ring carbon atoms, preferably three to about six carbon atoms, such as cyclopropyl, cyclobutyl, cyclopentyl and cyclohexyl. The term “haloalkyl” embraces radicals wherein any one or more of the alkyl carbon atoms is substituted with one or more halo groups, preferably selected from bromo, chloro and fluoro. Specifically embraced by the term “haloalkyl” are monohaloalkyl, dihaloalkyl and polyhaloalkyl groups. A monohaloalkyl group, for example, may have either a bromo, a chloro, or a fluoro atom within the group. Dihaloalkyl and polyhaloalkyl groups may be substituted with two or more of the same halo groups, or may have a combination of different halo groups. A dihaloalkyl group, for example, may have two fluoro atoms, such as difluoromethyl and difluorobutyl groups, or two chloro atoms, such as a dichloromethyl group, or one fluoro atom and one chloro atom, such as a fluoro-chloromethyl group. Examples of a polyhaloalkyl are trifluoromethyl, 1,1-difluoroethyl, 2,2,2-trifluoroethyl, perfluoroethyl and 2,2,3,3-tetrafluoropropyl groups. The term “difluoroalkyl” embraces alkyl groups having two fluoro atoms substituted on any one or two of the alkyl group carbon atoms. The terms “alkylol” and “hydroxyalkyl” embrace linear or branched alkyl groups having one to about ten carbon atoms any one of which may be substituted with one or more hydroxyl groups. The term “alkenyl” embraces linear or branched radicals having two to about twenty carbon atoms, preferably three to about ten carbon atoms, and containing at least one carbon-carbon double bond, which carbon-carbon double bond may have either cis or trans geometry within the alkenyl moiety. The term “alkynyl” embraces linear or branched radicals having two to about twenty carbon atoms, preferably two to about ten carbon atoms, and containing at least one carbon-carbon triple bond. The term “cycloalkenyl” embraces cyclic radicals having three to about ten ring carbon atoms including one or more double bonds involving adjacent ring carbons. The terms “alkoxy” and “alkoxyalkyl” embrace linear or branched oxy-containing radicals each having alkyl portions of one to about ten carbon atoms, such as methoxy group. The term “alkoxyalkyl” also embraces alkyl radicals having two or more alkoxy groups attached to the alkyl radical, that is, to form monoalkoxyalkyl and dialkoxyalkyl groups. The “alkoxy” or “alkoxyalkyl” radicals may be further substi-tuted with one or more halo atoms, such as fluoro, chloro or bromo, to provide haloalkoxy or haloalkoxyalkyl groups. The term “alkylthio” embraces radicals containing a linear or branched alkyl group, of one to about ten carbon atoms attached to a divalent sulfur atom, such as a methythio group. Preferred aryl groups are those consisting of one, two, or three benzene rings. The term “aryl” embraces aromatic radicals such as phenyl, naphthyl and biphenyl. The term “aralkyl” embraces aryl-substituted alkyl radicals such as benzyl, diphenylmethyl, triphenylmethyl, phenyl-ethyl, phenylbutyl and diphenylethyl. The terms “benzyl” and “phenylmethyl” are interchangeable. The terms “phenalkyl” and “phenylalkyl” are interchangeable. An example of “phenalkyl” is “phenethyl” which is interchangeable with “phenylethyl”. The terms “alkylaryl”, “alkoxyaryl” and “haloaryl” denote, respectively, the substitution of one or more “alkyl”, “alkoxy” and “halo” groups, respectively, substituted on an “aryl” nucleus, such as a phenyl moiety. The terms “aryloxy” and “arylthio” denote radicals respectively, provided by aryl groups having an oxygen or sulfur atom through which the radical is attached to a nucleus, examples of which are phenoxy and phenylthio. The terms “sulfinyl” and “sulfonyl”, whether used alone or linked to other terms, denotes, respectively, divalent radicals SO and SO₂. The term “aralkoxy”, alone or within another term, embraces an aryl group attached to an alkoxy group to form, for example, benzyloxy. The term “acyl” whether used alone, or within a term such as acyloxy, denotes a radical provided by the residue after removal of hydroxyl from an organic acid, examples of such radical being acetyl and benzoyl. “Lower alkanoyl” is an example of a more prefered sub-class of acyl. The term “amido” denotes a radical consisting of nitrogen atom attached to a carbonyl group, which radical may be further substituted in the manner described herein. The term “monoalkylaminocarbonyl” is interchangeable with “N-alkylamido”. The term “dialkylaminocarbonyl” is interchangeable with “N,N-dialkylamido”. The term “alkenylalkyl” denotes a radical having a double-bond unsaturation site between two carbons, and which radical may consist of only two carbons or may be further substituted with alkyl groups which may optionally contain additional double-bond unsaturation. The term “heteroaryl”, where not otherwised defined before, embraces aromatic ring systems containing one or two hetero atoms selected from oxygen, nitrogen and sulfur in a ring system having five or six ring members, examples of which are thienyl, furanyl, pyridinyl, thiazolyl, pyrimidyl and isoxazolyl. Such heteroaryl may be attached as a substituent through a carbon atom of the heteroaryl ring system, or may be attached through a carbon atom of a moiety substituted on a heteroaryl ring-member carbon atom, for example, through the methylene substituent of imidazolemethyl moiety. Also, such heteroaryl may be attached through a ring nitrogen atom as long as aromaticity of the heteroaryl moiety is preserved after attachment. For any of the foregoing defined radicals, preferred radicals are those containing from one to about ten carbon atoms.

Specific examples of alkyl groups are methyl, ethyl, n-propyl, isopropyl, n-butyl, sec-butyl, isobutyl, tert-butyl, n-pentyl, isopentyl, methylbutyl, dimethylbutyl and neopentyl. Typical alkenyl and alkynyl groups may have one unsaturated bond, such as an allyl group, or may have a plurality of unsaturated bonds, with such plurality of bonds either adjacent, such as allene-type structures, or in conjugation, or separated by several saturated carbons.

Also included in the combination of the invention are the isomeric forms of the above-described alpha-adrenergic modulating agents and the aldosterone receptor antagonist compounds, including diastereoisomers, regioisomers and the pharmaceutically-acceptable salts thereof. The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, p-hydroxybenzoic, salicyclic, phenylacetic, mandelic, embonic (pamoic), methansulfonic, ethanesulfonic, 2-hydroxyethanesulfonic, pantothenic, benzenesulfonic, toluenesulfonic, sulfanilic, mesylic, cyclohexylaminosulfonic, stearic, algenic, b-hydroxybutyric, malonic, galactaric and galacturonic acid. Suitable pharmaceutically-acceptable base addition salts include metallic salts made from aluminium, calcium, lithium, magnesium, potassium, sodium and zinc or organic salts made from N,N′-dibenzylethylenediamine, chloroprocaine, choline, diethanolamine, ethylenediamine, meglumine (N-methylgluca-mine) and procaine. All of these salts may be prepared by conventional means from the corresponding compound by reacting, for example, the appropriate acid or base with such compound.

Mechanism of Action

Without being held to a specific mechanism of action for the present combination therapy, it is hypothesized that the administration of these selected aldosterone receptor antagonists and alpha-adrenergic modulating agents in combination is effective because of the simultaneous and interrelated responses of tissues and/or organs to these two distinct classes of drugs: marked down-regulation of aldosterone-stimulated genetic effects in response to the aldosterone antagonist and: potent inhibition of alpha-1-adrenergic activity, in response to the alpha-1-adrenergic antagonist; or activation of the alpha-2-adrenergic receptor, in response to the alpha-2-adrenergic agonist. A non-limiting example of an interrelated mechanism would be a decrease in aldosterone induced vascular stiffness due to mechanical effects, such as fibrosis, combined with vasodilatory effects on vascular smooth muscle caused by alpha-1-adrenergic antagonism, or alpha-2-adrenergic agonism. Such an effect would provide a cooperative benefit to the therapeutic use of an aldosterone receptor antagonist.

Advantages of Combination Therapy

The selected aldosterone receptor antagonists and alpha-adrenergic modulating agents of the present invention act in combination to provide more than an additive benefit. For example, administration of an aldosterone receptor antagonist and alpha-1-adrenergic antagonist combination can result in the near-simultaneous reduction in pathogenic effects of multiple risk factors for atherosclerosis, such as high aldosterone levels, high blood pressure, endothelial dysfunction, plaque formation and rupture, etc.

The methods of this invention also provide for the effective prophylaxis and/or treatment of pathological conditions with reduced side effects compared to conventional methods known in the art. For example, administration of alpha-1-adrenergic antagonists can result in side effects such as, but not limited to, hypotension, increased heart rate, fluid retention, dizziness or syncope. Reduction of the alpha-1-adrenergic antagonist dose in the present combination therapy below conventional monotherapeutic doses will minimize, or even eliminate, the side-effect profile associated with the present combination therapy relative to the side-effect profiles associated with, for example, monotherapeutic administration of alpha-1-adrenergic antagonists. The side effects associated with alpha-1-adrenergic antagonists typically are dose-dependent and, thus, their incidence increases at higher doses. Accordingly, lower effective doses of alpha-1-adrenergic antagonists will result in fewer side effects than seen with higher doses of alpha-1-adrenergic antagonists in monotherapy or decrease the severity of such side-effects. In addition, the use of an aldosterone antagonist may provide a direct benefit in preventing or treating these side effects.

Other benefits of the present combination therapy include, but are not limited to, the use of a selected group of aldosterone receptor antagonists that provide a relatively quick onset of therapeutic effect and a relatively long duration of action. For example, a single dose of one of the selected aldosterone receptor antagonists may stay associated with the aldosterone receptor in a manner that can provide a sustained blockade of mineralocorticoid receptor activation. Another benefit of the present combination therapy includes, but is not limited to, the use of a selected group of aldosterone receptor antagonists, such as the epoxy-steroidal aldosterone antagonists exemplified by eplerenone, which act as highly selective aldosterone antagonists, with reduced side effects that can be caused by aldosterone antagonists that exhibit non-selective binding to non-mineralocorticoid receptors, such as androgen or progesterone receptors.

Further benefits of the present combination therapy include, but are not limited to, the use of the methods of this invention to treat individuals who belong to one or more specific ethnic groups that are particularly responsive to the disclosed therapeutic regimens. Thus, for example, individuals of African or Asian ancestry may particularly benefit from the combination therapy of an aldosterone antagonist and an alpha-adrenergic modulating agent to treat or prevent a cardiovascular disorder.

Subject Populations

Certain groups are more prone to disease modulating effects of aldosterone. Members of these groups that are sensitive to aldosterone are typically also salt sensitive, wherein individuals blood pressure generally rises and falls with increased and decreased sodium consumption, respectively. While the present invention is not to be construed as limited in practice to these groups, it is contemplated that certain subject groups may be particularly suited for therapy with the present invention. Accordingly, subjects who can benefit from treatment or prophylaxis in accordance with the method of the present invention are human subjects generally exhibiting one or more of the following characteristics:

(a) the average daily intake of sodium chloride by the subject is at least about 4 grams, particularly where this condition is satisfied over any one month interval for at least one or more monthly intervals over a given annual period. The average daily intake of sodium by the subject preferably is at least about 6 grams, more preferably at least about 8 grams, and still more preferably at least about 12 grams.

(b) the subject exhibits an increase in systolic blood pressure and/or diastolic blood pressure of at least about 5%, preferably at least about 7%, and more preferably at least about 10%, when daily sodium chloride intake by the subject is increased from less than about 3 g/day to at least about 10 g/day.

(c) the activities ratio of plasma aldosterone (ng/dL) to plasma renin (ng/ml/hr) in the subject is greater than about 30, preferably greater than about 40, more preferably greater than about 50; and still more preferably greater than about 60.

(d) the subject has low plasma renin levels; for example, the morning plasma renin activity in the subject is less than about 1.0 ng/dL/hr, and/or the active renin value in the subject is less than about 15 pg/mL.

(e) the subject suffers from or is susceptible to elevated systolic and/or diastolic blood pressure. In general, the systolic blood pressure (measured, for example, by seated cuff mercury sphygmomanometer) of the subject is at least about 130 mm Hg, preferably at least about 140 mm Hg, and more preferably at least about about 150 mm Hg, and the diastolic blood pressure (measured, for example, by seated cuff mercury sphygmomanometer) of the subject is at least about 85 mm Hg, preferably at least about 90 mm Hg, and more preferably at least about 100 mm Hg.

(f) the urinary sodium to potassium ratio (mmol/mmol) of the subject is less than about 6, preferably less than about 5.5, more preferably less than about 5, and still more preferably less than about 4.5.

(g) the urinary sodium level of the subject is at least 60 mmol per day, particularly where this condition is satisfied over any one month interval for at least one or more monthly intervals over a given annual period. The urinary sodium level of the subject preferably is at least about 100 mmol per day, more preferably at least about 150 mmol per day, and still more preferably 200 mmol per day.

(h) the plasma concentration of one or more endothelins, particularly plasma immunoreactive ET-1, in the subject is elevated. Plasma concentration of ET-1 preferably is greater than about 2.0 pmol/L, more preferably greater than about 4.0 pmol/L, and still more preferably greater than about 8.0 pmol/L.

(i) the subject has blood pressure that is substantially refractory to treatment with an ACE inhibitor; particularly a subject whose blood pressure is lowered less than about 8 mm Hg, preferably less than 5 mm Hg, and more preferably less than 3 mm Hg, in response to 10 mg/day enalapril compared to the blood pressure of the subject on no antihypertensive therapy.

(j) the subject has blood volume-expanded hypertension or blood volume-expanded borderline hypertenision, that is, hypertension wherein increased blood volume as a result of increased sodium retension contributes to blood pressure.

(k) the subject is a non-modulating individual, that is, the individual demonstrates a blunted positive response in renal blood flow rate and/or in adrenal production of aldosterone to an elevation in sodium intake or to angiotensin II administration, particularly when the response is less than the response of individuals sampled from the general geographical population (for example, individuals sampled from the subject's country of origin or from a country of which the subject is a resident), preferably when the response is less than 40% of the mean of the population, more preferably less than 30%, and more preferably still less than 20%.

(l) the subject has or is susceptible to renal dysfunction, particularly renal dysfunction selected from one or more members of the group consisting of reduced glomerular filtration rate, microalbuminuria, and proteinuria.

(m) the subject has or is susceptible to cardiovascular disease, particularly cardiovascular disease selected from one or more members of the group consisting of heart failure, left ventricular diastolic dysfunction, hypertrophic cardiomyopathy, and diastolic heart failure.

(n) the subject has or is susceptible to liver disease, particularly liver cirrhosis.

(o) the subject has or is susceptible to edema, particularly edema selected from one or more members of the group consisting of peripheral tissue edema, hepatic or splenic congestion, liver ascites, and respiratory or lung congestion.

(p) the subject has or is susceptible to insulin resistance, particularly Type I or Type II diabetes mellitus, and/or glucose sensitivity.

(q) the subject is at least 55 years of age, preferably at least about 60 years of age, and more preferably at least about 65 years of age.

(r) the subject is, in whole or in part, a member of at least one ethnic group selected from the Asian (particularly from the Japanese) ethnic group, the American Indian ethnic group, and the Black ethnic group.

(s) the subject has one or more genetic markers associated with salt sensitivity.

(t) the subject is obese, preferably with greater than 25% body fat, more preferably with greater than 30% body fat, and even more preferably with greater than 35% body fat.

(u) the subject has one or more 1^{st}, 2^{nd}, or 3^{rd} degree relatives who are or were salt sensitive, wherein 1^{st} degree relatives means parents or relatives sharing one or more of the same parents, 2^{nd} degree relatives means grandparents and relatives sharing one or more of the same grandparents, and 3^{rd} degree relatives means great-grandparents and relatives sharing one or more of the same great-grandparents. Preferably, such individuals have four or more salt sensitive 1^{st}, 2^{nd}, or 3^{rd} degree relatives; more preferably, eight or more such relatives; even more preferably, 16 or more such relatives; and even more preferably still, 32 or more such relatives.

Unless otherwise indicated to the contrary, the values listed above preferably represent an average value, more preferably a daily average value based on at least two measurements.

Preferably, the subject in need of treatment satisfies at least two or more of the above-characteristics, or at least three or more of the above-characteristics, or at least four or more of the above-characteristics.

Biological Evaluation

Human congestive heart failure (CHF) is a complex condition usually initiated by vascular hypertension or a myocardial infarction (MI). In order to determine the probable effectiveness of a combination therapy for CHF, it is important to determine the potency of components in several assays. Accordingly, in Assays “A” and “B”, the alpha-1-adrenergic antagonist activity can be determined. In Assays “C” and “D” a method is described for evaluating a combination therapy of the invention, namely, an alpha-1-adrenergic antagonist and an epoxy-steroidal aldosterone receptor antagonist. The efficacy of the individual drugs, eplerenone and an alpha-1-adrenergic antagonist, and of these drugs given together at various doses, are evaluated in rodent models of hypertension and CHF using surgical alterations to induce either hypertension or an MI. The methods of such assays are described below.

In addition, clinical trials can be used to evaluate aldosterone antagonist therapy in humans. Numerous examples of such therapeutic tests have been published, including those of the RALES 003 study described in American Journal of Cardiology 78, 902-907 (1996) or the RALES 004 study described in New England Journal of Medicine 341, 709-717 (1999).

Assay A: In Vitro Vascular Smooth Muscle-Response

Thoracic aortas, removed from male Sprague-Dawley rats (350-550 g), are dissected free from surrounding connective tissue, and cut into ring segments each about 2-3 mm long. Smooth muscle rings are mounted for isometric tension recording in an organ bath filled with 10 mL of Krebs-Henseleit (K—H) solution pH 7.4). This bathing solution is maintained at 37C. and bubbled with 95% O₂/5% CO₂. The strips are stretched to a tension of 2 g and allowed to equilibrate. Isometric tension changes are monitored using an isometric transducer and recorded on a potentiometric recorder. A precontraction is produced by adding a catecholamine or by changing the solution to 30 mM K⁺. Contraction is maintained for 30 min, and the preparation wahed with Krebs-Henseleit solution. After sixty minutes contraction is induced in the same manner as described above. Subsequently a test compound is added to obtain a concentration-response curve. Taking the precontraction value as 100%, the concentration of the drug at which the contraction is relaxed to 50% is the IC₅₀.

Assay B: In Vivo Intragastric Pressor Assay Response

Male Sprague-Dawley rats weighing 225-300 grams are anesthetized with methohexital (30 mg/kg, i.p.) and catheters were implanted into the femoral artery and vein. The catheters are tunneled subcutaneously to exit dorsally, posterior to the head and between the scapulae. The catheters are filled with heparin (1000 units/ml of saline). The rats are returned to their cage and allowed regular rat chow and water ad libitum. After full recovery from surgery (3-4 days), rats are placed in Lucite holders and the arterial line is connected to a pressure transducer. Arterial pressure is recorded on a Gould polygraph (mmHg). Epinephrine or norepinephrine is administered as a 30 ng/kg bolus via the venous catheter delivered in a 50 μl volume with a 0.2 ml saline flush. The pressor response in mm Hg is measured by the difference from pre-injection arterial pressure to the maximum pressure achieved. The catecholamine injection is repeated every 10 minutes until three consecutive injections yield responses within 4 mmHg of each other. These three responses are then averaged and represent the control response to catecholamines. The test compound is suspended in 0.5% methylcellulose in water and is administered by gavage. The volume administered is 2 ml/kg body weight. Catecholamine bolus injections are given at 30, 45, 60, 75, 120, 150, and 180 minutes after gavage. The pressor response to the catecholamine is measured at each time point. The rats are then returned to their cage for future testing. A minimum of 3 days is allowed between tests. Percent inhibition is calculated for each time point following gavage by the following formula:

[(Control Response−Response at time point)/Control Response]×100.

Assay “C”: Hypertensive Rat Model

Male rats are made hypertensive by placing a silver clip with an aperture of 240 microns on the left renal artery, leaving the contralateral kidney untouched. Sham controls undergo the same procedure but without attachment of the clip. One week prior to the surgery, animals to be made hypertensive are divided into separate groups and drug treatment is begun. Groups of animals are administered vehicle, alpha-1-adrenergic antagonist (or alpha-2-adrenergic agonist) alone, eplerenone alone, and combinations of an alpha-adrenergic modulating agent and eplerenone at various doses:

| | |
|---|---|
| Alpha-1- | Combination of |

| Adrenergic | | Alpha-1-Adrenergic | |
|---|---|---|---|
| Antagonist | Eplerenone | Antagonist | Eplerenone |
| (mg/kg/day) | (mg/kg/day) | (mg/kg/day) | (mg/kg/day) |
| 3 | 5 | 3 | 5 |
| | 20 | 3 | 20 |
| | 50 | 3 | 50 |
| | 100 | 3 | 100 |
| | 200 | 3 | 200 |
| 10 | 5 | 10 | 5 |
| | 20 | 10 | 20 |
| | 50 | 10 | 50 |
| | 100 | 10 | 100 |
| | 200 | 10 | 200 |
| 30 | 5 | 30 | 5 |
| | 20 | 30 | 20 |
| | 50 | 30 | 50 |
| | 100 | 30 | 100 |
| | 200 | 30 | 200 |

After 12 to 24 weeks, systolic and diastolic blood pressure, left ventricular end diastolic pressure, left ventricular dP/dt, and heart rate are evaluated. The hearts are removed, weighed, measured and fixed in formalin. Collagen content of heart sections are evaluated using computerized image analysis of picrosirius stained sections. It would be expected that rats treated with a combination therapy of either an alpha-1-adrenergic antagonist or an alpha-2-adrenergic agonist and eplerenone components, will show improvements in cardiac performance, as compared to rats treated with either component alone.

Assay “D”: Myocardial Infarction Rat Model:

Male rats are anesthetized and the heart is exteriorized following a left sided thoracotomy. The left anterior descenting coronary artery is ligated with a suture. The thorax is closed and the animal recovers. Sham animals have the suture passed through without ligation. One week prior to the surgery, animals to undergo infarction are divided into separate groups and drug treatment is begun. Groups of animals are administered vehicle, an alpha-adrenergic modulating agent alone, eplerenone alone, and combinations of an alpha-adrenergic modulating agent and eplerenone, at various doses. The following table lists a protocol for testing the efficacy of an alpha-1-adrenergic antagonist and eplerenone, both administered separately or in combination:

| Alpha-1- | | Combination of | |
|---|---|---|---|
| Adrenergic | | Alpha-1-Adrenergic | |
| Antagonist | Eplerenone | Antagonist | & Eplerenone |
| (mg/kg/day) | (mg/kg/day) | (mg/kg/day) | (mg/kg/day) |
| 3 | 5 | 3 | 5 |
| | 20 | 3 | 20 |
| | 50 | 3 | 50 |
| | 100 | 3 | 100 |
| | 200 | 3 | 200 |
| 10 | 5 | 10 | 5 |
| | 20 | 10 | 20 |
| | 50 | 10 | 50 |
| | 100 | 10 | 100 |
| | 200 | 10 | 200 |
| 30 | 5 | 30 | 5 |
| | 20 | 30 | 20 |
| | 50 | 30 | 50 |
| | 100 | 30 | 100 |
| | 200 | 30 | 200 |

After six weeks, systolic and diastolic blood pressure, left ventricular end diastolic pressure, left ventricular dP/dt, and heart rate are evaluated. The hearts are removed, weighed, measured and fixed in formalin. Collagen content of heart sections are evaluated using computerized image analysis of picrosirius stained sections. It would be expected that rats treated with a combination therapy of either an alpha-1-adrenergic antagonist or an alpha-2-adrenergic agonist and eplerenone components, will show improvements in cardiac performance, as compared to rats treated with either component alone.

Administration of the alpha-adrenergic modulating agent and the aldosterone receptor antagonist may take place sequentially in separate formulations, or may be accomplished by simultaneous administration in a single formulation or separate formulations.

Administration may be accomplished by oral route, or by intravenous, intramuscular or subcutaneous injections. The formulation may be in the form of a bolus, or in the form of aqueous or non-aqueous isotonic sterile injection solutions or suspensions. These solutions and suspensions may be prepared from sterile powders or granules having one or more pharmaceutically-acceptable carriers or diluents, or a binder such as gelatin or hydroxypropyl-methyl cellulose, together with one or more of a lubricant, preservative, surface-active or dispersing agent.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. These may contain, for example, an amount of each active ingredient from about 1 mg to about 1000 mg, or about 5 mg to about 500 mg, or about 10 mg to about 250 mg, or about 25 mg to about 150 mg. A suitable daily dose for a mammal may vary widely depending on the condition of the patient and other factors. However, a dose of from about 0.01 to 30 mg/kg body weight, particularly from about 1 to 15 mg/kg body weight, may be appropriate.

The active ingredients may also be administered by injection as a composition wherein, for example, saline, dextrose or water may be used as a suitable carrier. A suitable daily dose of each active component is from about 0.01 to 15 mg/kg body weight injected per day in multiple doses depending on the disease being treated. A preferred daily dose would be from about 1 to 10 mg/kg body weight. Compounds indicated for prophylactic therapy will preferably be administered in a daily dose generally in a range from about 0.1 mg to about 15 mg per kilogram of body weight per day. A more preferred dosage will be a range from about 1 mg to about 15 mg per kilogram of body weight. Most preferred is a dosage in a range from about 1 to about 10 mg per kilogram of body weight per day. A suitable dose can be administered, in multiple sub-doses per day. These sub-doses may be administered in unit dosage forms. Typically, a dose or sub-dose may contain from about 1 mg to about 100 mg of active compound per unit dosage form. A more preferred dosage will contain from about 2 mg to about 50 mg of active compound per unit dosage form. Most preferred is a dosage form containing from about 3 mg to about 25 mg of active compound per unit dose.

In combination therapy, the alpha-1-adrenergic antagonist may be present in a range of doses, depending on the particular antagonist used, inherent potency, bioavailabilty and metabolic lability of the composition and whether it has been formulated for immediate release or extended release. Non-limiting examples of dose form ranges for specific alpha-1-adrenergic antagonists are listed below:

| Component | | ILLUSTRATIVE | ILLUSTRATIVE | |
|---|---|---|---|---|
| Number | COMPOUND | DOSAGE FORM | DOSE | |
| A-1 | amosulalol | | | |
| A-2 | arotinolol | | | |
| A-3 | dapiprazole | solution/drops | 0.5% | |
| A-4 | doxazosin | tablet/oral | 1 mg-8 mg | |
| A-5 | fenspirlde | | | |
| A-6 | indoramin | | | |
| A-7 | labetalol | tablet/oral; | 100 mg-300 mg; | |
| | | injectable/ | 5 | mg/ml |
| | | injection | | |
| A-8 | naftopidil | | | |
| A-9 | nicergoline | | | |
| A-10 | prazosin | capsule/oral | 0.5 mg-5 mg | |
| A-11 | tamsulosin | capsule/oral | 0.4 | mg |
| A-12 | tolazoline | injectable/ | 25 | mg/ml |
| | | injection | | |
| A-13 | trimazosin | | | |
| A-14 | yohimbine | | | |
| A-15 | phenoxybenzamine | capsule/oral | 10 | mg |
| A-16 | phentolamine | injectable/ | 5 | mg |
| | | injection | | |

| | | | |
|---|---|---|---|
| A-17 | terazosin | capsule/oral | 1 mg-10 mg |
| A-18 | bunazosin | | |
| A-19 | urapidil | | |
| A-20 | alfuzosin | | |
| A-21 | ketanserin | | |
| A-22 | monatepil | | |
| A-23 | Sun 9221 | | |
| A-24 | S-2150 | | |

In combination therapy, the alpha-2-adrenergic agonist may be present in a range of doses, depending on the particular agonist used, inherent potency, bioavailabilty and metabolic lability of the composition and whether it has been formulated for immediate release or extended release. Non-limiting examples of dose form ranges for specific alpha-2-adrenergic agonists are listed below:

| Component | | ILLUSTRATIVE | ILLUSTRATIVE |
|---|---|---|---|
| Number | COMPOUND | DOSAGE FORM | DOSE |
| A-25 | clonidine | tablet/oral; | 0.1-15 mg; |
| | | solution/drops; | 0.5%; |
| | | film; | 0.1-0.3 mg; |
| | | injectable/ | 0.1 mg |
| | | injection | |
| A-26 | apraclonidine | solution/drops | 0.5-1% |
| A-27 | guanfacine | tablet/oral | 1-2 mg |
| A-28 | guanabenz | tablet/oral | 4-8 mg |
| A-29 | guanfacil | | |
| A-30 | rilmenidine | | |
| A-31 | maxonidine | | |

One of ordinary skill in the art will be capable of using these dose ranges as a suitable starting point to administer this therapy, after which the dose may be titrated up or down, depending on the response of the subject being treated.

The dosage regimen for treating a disease condition with the combination therapy of this invention is selected in accordance with a variety of factors, including the type, age, weight, sex and medical condition of the patient, the severity of the disease, the route of administration, and the particular compound employed, and thus may vary widely.

Below, non-limiting examples of combinations of the present invention are listed wherein the combination comprises a first amount of an aldosterone receptor antgonist and a second amount of an alpha-adrenergic modulating agent wherein the first amount and second amount together comprise a therapeutically-effective amount of an aldosterone receptor antagonist and an alpha-adrenergic modulating-agent:

| EXAMPLE | COMPONENT 1 | COMPONENT 2 |
|---|---|---|
| 1 | Eplerenone | A-1 |
| 2 | Eplerenone | A-2 |
| 3 | Eplerenone | A-3 |
| 4 | Eplerenone | A-4 |
| 5 | Eplerenone | A-5 |
| 6 | Eplerenone | A-6 |
| 7 | Eplerenone | A-7 |
| 8 | Eplerenone | A-8 |
| 9 | Eplerenone | A-9 |
| 10 | Eplerenone | A-10 |
| 11 | Eplerenone | A-11 |
| 12 | Eplerenone | A-12 |
| 13 | Eplerenone | A-13 |
| 14 | Eplerenone | A-14 |
| 15 | Eplerenone | A-15 |
| 16 | Eplerenone | A-16 |
| 17 | Eplerenone | A-17 |
| 18 | Eplerenone | A-18 |
| 19 | Eplerenone | A-19 |
| 20 | Eplerenone | A-20 |
| 21 | Eplerenone | A-21 |
| 22 | Eplerenone | A-22 |
| 23 | Eplerenone | A-23 |
| 24 | Eplerenone | A-24 |
| 25 | Eplerenone | A-25 |
| 26 | Eplerenone | A-26 |
| 27 | Eplerenone | A-27 |
| 28 | Eplerenone | A-28 |
| 29 | Eplerenone | A-29 |
| 30 | Eplerenone | A-30 |
| 31 | Eplerenone | A-31 |
| 32 | Spironolactone | A-1 |
| 33 | Spironolactone | A-2 |
| 34 | Spironolactone | A-3 |
| 35 | Spironolactone | A-4 |
| 36 | Spironolactone | A-5 |
| 37 | Spironolactone | A-6 |
| 38 | Spironolactone | A-7 |
| 39 | Spironolactone | A-8 |
| 40 | Spironolactone | A-9 |
| 41 | Spironolactone | A-10 |
| 42 | Spironolactone | A-11 |
| 43 | Spironolactone | A-12 |
| 44 | Spironolactone | A-13 |
| 45 | Spironolactone | A-14 |
| 46 | Spironolactone | A-15 |
| 47 | Spironolactone | A-16 |
| 48 | Spironolactone | A-17 |
| 49 | Spironolactone | A-18 |
| 50 | Spironolactone | A-19 |
| 51 | Spironolactone | A-20 |
| 52 | Spironolactone | A-21 |
| 53 | Spironolactone | A-22 |
| 54 | Spironolactone | A-23 |
| 55 | Spironolactone | A-24 |
| 56 | Spironolactone | A-25 |
| 57 | Spironolactone | A-26 |
| 58 | Spironolactone | A-27 |
| 59 | Spironolactone | A-28 |
| 60 | Spironolactone | A-29 |
| 61 | Spironolactone | A-30 |
| 62 | Spironolactone | A-31 |

In combination therapy, the aldosterone receptor antagonist may be present in an amount in a range from about 5 mg to about 400 mg, and the alpha-adrenergic modulating agent may be present in an amount in a range from about 1 mg to about 200 mg, which represents aldosterone antagonist-to-alpha-adrenergic modulating agent ratios ranging from about 400:1 to about 1:40.

In another embodiment, the aldosterone receptor antagonist may be present in an amount in a range from about 10 mg to about 200 mg, and the alpha-adrenergic modulating agent may be present in an amount in a range from about 5 mg to about 100 mg, which represents aldosterone antagonist-to-alpha-adrenergic modulating agent ratios ranging from about 40:1 to about 1:10.

In another embodiment, the aldosterone receptor antagonist may be present in an amount in a range from about 20 mg to about 100 mg, and the alpha-adrenergic modulating agent may be present in an amount in a range from about 10 mg to about 80 mg, which represents aldosterone antagonist-to-alpha-adrenergic modulating agent ratios ranging from about 10:1 to about 1:4.

In another embodiment the alpha-adrenergic modulating agent and the aldosterone receptor antagonist are present in the combination in a weight ratio from about one-to-one to about one-to-twenty, of the alpha-adrenergic modulating agent to the aldosterone receptor antagonist.

In another embodiment the alpha-adrenergic modulating agent and the aldosterone receptor antagonist are present in the combination in a weight ratio from about one-to-five to about one-to-fifteen, of the alpha-adrenergic modulating agent to the aldosterone receptor antagonist.

In another embodiment the alpha-adrenergic modulating agent and the aldosterone receptor antagonist are present in the combination in a weight ratio of about one-to-ten, of the alpha-adrenergic modulating agent to the aldosterone receptor antagonist.

For therapeutic purposes, the active components of this combination therapy invention are ordinarily combined with one or more adjuvants appropriate to the indicated route of administration. If administered per os, the components may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The components may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

In another embodiment the active components of this combination therapy are combined, with one or more pharmaceutically acceptable adjuvants, to form tablet or capsule (composition) which exhibits a release profile, determined using a suitable release profile test, in which more than about 20% by weight of the aldosterone receptor antagonist is released from the composition at about four hours after initiation of the test. A non-limiting example of a suitable release profile test is a test conducted according to U.S. Pharmacopeia 24, incorporated herein by reference, Test No. 711, using apparatus 2 at 50 rpm, with an aqueous dissolution medium containing 1% sodium dodecyl sulfate at 37° C., wherein release is measured by dissolution of the aldosterone receptor antagonist in the medium.

The present invention further comprises kits that are suitable for use in performing the methods of treatment and/or prophylaxis described above. In one embodiment, the kit contains a first dosage form comprising one or more of the aldosterone receptor antagonists previously identified and a second dosage form comprising an alpha-adrenergic modulating agent identified in Tables 2 and 3 and designated by Component Number as A-1 to A-31 above, in quantities sufficient to carry out the methods of the present invention. Preferably, the first dosage form and the second dosage form together comprise a therapeutically effective amount of the compounds. In another embodiment, the kit further comprises written instructions stating how the contents of the kit can be used by the subject. The written instructions will be useful, for example, for the subject to obtain a therapeutic effect without inducing unwanted side-effects. In another embodiment the written instructions comprise all or a part of the product label approved by a drug regulatory agency for the kit.

Crystalline Forms of Active Compounds

It is particularly useful to select a form of each active compound that is easily handled, reproducible in form, easily prepared, stable and which is non-hygroscopic. By way of illustration and not limitation, several crystalline forms have been identified for the aldosterone antagonist eplerenone. These include Form H, Form L, various crystalline solvates and amorphous eplerenone. These forms, methods to make these forms and use of these forms in preparing compositions and medicaments, are disclosed in the following publications, incorporated herein by reference: WO 01/41535 and WO 01/42272.

What is claimed is:

1. A combination for the treatment or prevention of a cardiovascular disorder comprising a first amount of an aldosterone receptor antagonist and a second amount of an alpha-adrenergic modulating agent, wherein said first amount and said second amount together comprise a therapeutically-effective amount of said aldosterone receptor antagonist and said alpha-adrenergic modulating agent, and wherein said alpha-adrenergic modulating agent is selected from the group consisting of amosulalol, arotinolol, dapiprazole, fenspirlde, indoramin, naftopidil, nicergoline, tamsulosin, tolazoline, trimazosin, yohimbine, phenoxybenzamine, phentolamine, bunazosin, urapidil, alfuzosin, ketanserin, monatepil, SUN 9221, S-2150, apraclonidine, guanfacil, rilmenidine, and moxonidine.

2. The combination of claim 1 wherein said alpha-adrenergic modulating agent is selected from the group consisting of dapiprazole, tamsulosin, tolazoline, phenoxybenzamine, phentolamine, and apraclonidine.

3. The combination of claim 1 wherein said alpha-adrenergic modulating agent is selected from the group consisting of amosulalol, arotinolol, fenspirlde, indoramin, naftopidil, nicergoline, trimazosin, yohimbine, bunazosin, urapidil, alfuzosin, ketanserin, monatepil, SUN 9221, S-2150, guanfacil, rilmenidine, and moxonidine.

4. A pharmaceutical composition for the treatment or prevention of a cardiovascular disorder comprising a first amount of an aldosterone receptor antagonist, a second amount of an alpha-adrenergic modulating agent, and one or more pharmaceutically acceptable carrier materials, wherein said first amount and said second amount together comprise a therapeutically-effective amount of said aldosterone receptor antagonist and said alpha-adrenergic modulating agent, and wherein said alpha-adrenergic modulating agent is selected from the group consisting of amosulalol, arotinolol, dapiprazole, fenspirlde, indoramin, naftopidil, nicergoline, tamsulosin, tolazoline, trimazosin, yohimbine, phenoxybenzamine, phentolamine, bunazosin, urapidil, alfuzosin, ketanserin, monatepil, SUN 9221, S-2150, apraclonidine, guanfacil, rilmenidine, and moxonidine.

5. The pharmaceutical composition of claim 4 wherein said alpha-adrenergic modulating agent is selected from the group consisting of dapiprazole, tamsulosin, tolazoline, phenoxybenzamine, phentolamine, and apraclonidine.

6. The pharmaceutical composition of claim 4 wherein said alpha-adrenergic modulating agent is selected from the group consisting of amosulalol, arotinolol, fenspirlde, indoramin, naftopidil, nicergoline, trimazosin, yohimbine, bunazosin, urapidil, alfuzosin, ketanserin, monatepil, SUN 9221, S-2150, guanfacil, rilmenidine, and moxonidine.

7. The pharmaceutical composition of claim 4 wherein said aldosterone receptor antagonist is an epoxy-steroidal aldosterone receptor antagonist.

8. The pharmaceutical composition of claim 7 wherein said epoxy-steroidal aldosterone receptor antagonist has an epoxy moiety fused to the “C” ring of the steroidal nucleus of a 20-spiroxane compound.

9. The pharmaceutical composition of claim 8 wherein said 20-spiroxane compound is characterized by the presence of a 9α-,11α-substituted epoxy moiety.

10. The pharmaceutical composition of claim 7 wherein said epoxy-steroidal aldosterone receptor antagonist is selected from the group consisting of:
Eplerenone;
Pregn-4-ene-7,21-dicarboxylic acid, 9,11-epoxy-17-hydroxy-3-oxo-dimethyl ester, (7α,11α,17β)-;
3′H-cyclopropa[6,7]pregna-4,6-diene-21-carboxylic acid, 9,11-epoxy-6,7-dihydro-17-hydroxy-3-oxo-, γ-lactone, (6β,7β,11α,17β)-;
Pregn-4-ene-7,21-dicarboxylic acid, 9,11-epoxy-17-hydroxy-3-oxo, 7-(1-methylethyl) ester, monopotassium salt, (7α,11α,17β)-;
Pregn-4-ene-7,21-dicarboxylic acid, 9,11-epoxy-17-hydroxy-3-oxo-, 7-methyl ester, monopotassium salt, (7α,11α,17β)-;
3′H-cyclopropa[6,7]pregna-1,4,6-triene-21-carboxylic acid, 9,11-epoxy-6,7-dihydro-17-hydroxy-3-oxo-, γ-lactone, (6β,7β,11α)-;
3′H-cyclopropa[6,7]pregna-4,6-diene-21-carboxylic acid, 9,11-epoxy-6,7-dihydro-17-hydroxy-3-oxo-, methyl ester, (6β,7β,11α,17β)-;
3′H-cyclopropa[6,7]pregna-4,6-diene-21-carboxylic acid, 9,11-epoxy-6,7-dihydro-17-hydroxy-3-oxo-, monopotassium salt, (6β,7β,11α,17β)-;
3′H-cyclopropa[6,7]pregna-4,6-diene-21-carboxylic acid, 9,11-epoxy-6,7-dihydro-17-hydroxy-3-oxo-, γ-lactone, (6β,7β,11α,17β)-;
Pregn-4-ene-7,21-dicarboxylic acid, 9,11-epoxy-17-hydroxy-3-oxo-, γ-lactone, ethyl ester, (7α,11α,17β)-; and
Pregn-4-ene-7,21-dicarboxylic acid, 9,11-epoxy-17-hydroxy-3-oxo-, γ-lactone, 1-methylethyl ester, (7α,11α,17β)-.

11. The pharmaceutical composition of claim 7 wherein said epoxy-steroidal aldosterone receptor antagonist is eplerenone.

12. The pharmaceutical composition of claim 11 wherein said alpha-adrenergic modulating agent is selected from the group consisting of amosulalol, arotinolol, dapiprazole, fenspirlde, indoramin, naftopidil, nicergoline, tamsulosin, tolazoline, trimazosin, yohimbine, phenoxybenzamine, phentolamine, bunazosin, urapidil, alfuzosin, ketanserin, monatepil, SUN 9221, and S-2150.

13. The pharmaceutical composition of claim 12 wherein said alpha-adrenergic modulating agent and said aldosterone receptor antagonist are present in said combination in a weight ratio range from about one-to-one to about one-to-twenty of said alpha-adrenergic modulating agent to said aldosterone receptor antagonist.

14. The pharmaceutical composition of claim 13 wherein said weight ratio range is from about one-to-five to about one-to-fifteen.

15. The pharmaceutical composition of claim 13 wherein said weight ratio is about one-to-ten.

16. The pharmaceutical composition of claim 12 wherein said first amount of eplerenone is between about 0.1 mg to about 400 mg.

17. The pharmaceutical composition of claim 11 wherein said alpha-adrenergic modulating agent is selected from the group consisting of apraclonidine, guanfacil, rilmenidine, and moxonidine.

18. The pharmaceutical composition of claim 17 wherein said alpha-adrenergic modulating agent and said aldosterone receptor antagonist are present in said combination in a weight ratio range from about one-to-one to about one-to-twenty of said alpha-adrenergic modulating agent to said aldosterone receptor antagonist.

19. The pharmaceutical composition of claim 18 wherein said weight ratio range is from about one-to-five to about one-to-fifteen.

20. The pharmaceutical composition of claim 18 wherein said weight ratio is about one-to-ten.

21. The pharmaceutical composition of claim 17 wherein said first amount of eplerenone is between about 0.1 mg to about 400 mg.

22. The pharmaceutical composition of claim 4 wherein said aldosterone antagonist is spironolactone.

23. The pharmaceutical composition of claim 22 wherein said alpha-adrenergic modulating agent is selected from the group consisting of amosulalol, arotinolol, dapiprazole, fenspirlde, indoramin, naftopidil, nicergoline, tamsulosin, tolazoline, trimazosin, yohimbine, phenoxybenzamine, phentolamine, bunazosin, urapidil, alfuzosin, ketanserin, monatepil, SUN 9221, and S-2150.

24. The pharmaceutical composition of claim 22 wherein said alpha-adrenergic modulating agent is selected from the group consisting of apraclonidine, guanfacil, rilmenidine, and moxonidine.

25. The pharmaceutical composition of claim 22 wherein said alpha-adrenergic modulating agent and said aldosterone receptor antagonist are present in said combination in a weight ratio range from about one-to-one to about one-to-twenty of said alpha-adrenergic modulating agent to said aldosterone receptor antagonist.

26. A method for the treatment or prevention of a cardiovascular disorder in a subject susceptible to or afflicted with such disorder comprising administering to the subject a first amount of an aldosterone receptor antagonist and a second amount of an alpha-adrenergic modulating agent, wherein said first and second amount together comprise a therapeutically-effective amount of said aldosterone receptor antagonist and said alpha-adrenergic modulating agent, and wherein said alpha-adrenergic modulating agent is selected from the group consisting of amosulalol, arotinolol, dapiprazole, fenspirlde, indoramin, naftopidil, nicergoline, tamsulosin, tolazoline, trimazosin, yohimbine, phenoxybenzamine, phentolamine, bunazosin, urapidil, alfuzosin, ketanserin, monatepil, SUN 9221, S-2150, apraclonidine, guanfacil, rilmenidine, and moxonidine.

27. The method of claim 26, wherein said cardiovascular disorder is selected from the group consisting of hypertension, heart failure, cirrhosis and ascites.

28. The method of claim 26, wherein said cardiovascular disorder is hypertension.

29. The method of claim 26, wherein said cardiovascular disorder is heart failure.

30. The method of claim 26 wherein said aldosterone receptor antagonist and said alpha-adrenergic modulating agent are administered in a sequential manner.

31. The method of claim 26 wherein said aldosterone receptor antagonist and said alpha-adrenergic modulating agent are administered in a substantially simultaneous manner.

32. The method of claim 26 wherein said aldosterone receptor antagonist is an epoxy-steroidal aldosterone receptor antagonist.

33. The method of claim 32 wherein said epoxy-steroidal aldosterone receptor antagonist has an epoxy moiety fused to the “C” ring of the steroidal nucleus of a 20-spiroxane compound.

34. The method of claim 33 wherein said 20-spiroxane compound is characterized by the presence of a 9α-,11α-substituted epoxy moiety.

35. The method of claim 32 wherein said epoxy-steroidal aldosterone receptor antagonist is selected from the group consisting of:
Eplerenone;
Pregn-4-ene-7,21-dicarboxylic acid, 9,11-epoxy-17-hydroxy-3-oxo-dimethyl ester, (7α,11α,17β)-;
3′H-cyclopropa[6,7]pregna-4,6-diene-21-carboxylic acid, 9,11-epoxy-6,7-dihydro-17-hydroxy-3-oxo-, γ-lactone, (6β,7β,11α,17β)-;
Pregn-4-ene-7,21-dicarboxylic acid, 9,11-epoxy-17-hydroxy-3-oxo, 7-(1-methylethyl) ester, monopotassium salt, (7α,11α,17β)-;
Pregn-4-ene-7,21-dicarboxylic acid, 9,11-epoxy-17-hydroxy-3-oxo-, 7-methyl ester, monopotassium salt, (7α,11α,17β)-;
3′H-cyclopropa[6,7]pregna-1,4,6-triene-21-carboxylic acid, 9,11-epoxy-6,7-dihydro-17-hydroxy-3-oxo-, γ-lactone, (6β,7β,11α)-;
3′H-cyclopropa[6,7]pregna-4,6-diene-21-carboxylic acid, 9,11-epoxy-6,7-dihydro-17-hydroxy-3-oxo-, methyl ester, (6β,7β,11α,17β)-;
3′H-cyclopropa[6,7]pregna-4,6-diene-21-carboxylic acid, 9,11-epoxy-6,7-dihydro-17-hydroxy-3-oxo-, monopotassium salt, (6β,7β,11α,17β)-;
3′H-cyclopropa[6,7]pregna-4,6-diene-21-carboxylic acid, 9,11-epoxy-6,7-dihydro-17-hydroxy-3-oxo-, γ-lactone, (6β,7β,11α,17β)-;
Pregn-4-ene-7,21-dicarboxylic acid, 9,11-epoxy-17-hydroxy-3-oxo-, γ-lactone, ethyl ester, (7α,11α,17β)-; and
Pregn-4-ene-7,21-dicarboxylic acid, 9,11-epoxy-17-hydroxy-3-oxo-, γ-lactone, 1-methylethyl ester, (7α,11α,17β.

36. The method of claim 32 wherein said epoxy-steroidal aldosterone receptor antagonist is eplerenone.

37. The method of claim 36 wherein said alpha-adrenergic modulating agent is selected from the group consisting of amosulalol, arotinolol, dapiprazole, fenspirlde, indoramin, naftopidil, nicergoline, tamsulosin, tolazoline, trimazosin, yohimbine, phenoxybenzamine, phentolamine, bunazosin, urapidil, alfuzosin, ketanserin, monatepil, SUN 9221, and S-2150.

38. The method of claim 36 wherein said alpha-adrenergic modulating agent is selected from the group consisting of apraclonidine, guanfacil, rilmenidine, and moxonidine.

39. The method of claim 36 wherein said alpha-adrenergic modulating agent and said aldosterone receptor antagonist are administered in a weight ratio range from about one-to-one to about one-to-twenty of said alpha-adrenergic modulating agent to said aldosterone receptor antagonist.

40. The method of claim 39 said weight ratio range is from about one-to-five to about one-to-fifteen.

41. The method of claim 39 wherein said weight ratio is about one-to-ten.

42. The method of claim 36 wherein said eplerenone is administered in a daily dose range from about 0.1 mg to about 400 mg.

43. The method of claim 36 wherein said eplerenone is administered in a daily dose range from about 1 mg to about 200 mg.

44. The method of claim 36 wherein said eplerenone is administered in a daily dose range from about 10 mg to about 100 mg.

45. The method of claim 36 wherein said eplerenone is administered in a daily dose selected from the group consisting of 25 mg, 50 mg and 100 mg.

46. The method of claim 26 wherein said aldosterone antagonist is spironolactone.

47. The method of claim 46 wherein said alpha-adrenergic modulating agent is selected from the group consisting of amosulalol, arotinolol, dapiprazole, fenspirlde, indoramin, naftopidil, nicergoline, tamsulosin, tolazoline, trimazosin, yohimbine, phenoxybenzamine, phentolamine, bunazosin, urapidil, alfuzosin, ketanserin, monatepil, SUN 9221, and S-2150.

48. The method of claim 46 wherein said alpha-adrenergic modulating agent is selected from the group consisting of apraclonidine, guanfacil, rilmenidine, and moxonidine.

49. The method of claim 46 wherein said alpha-adrenergic modulating agent and said aldosterone receptor antagonist are administered in a weight ratio range from about one-to-one to about one-to-twenty of said alpha-adrenergic modulating agent to said aldosterone receptor antagonist.

50. A pharmaceutical composition for the treatment or prevention of a cardiovascular disorder comprising a first amount of an aldosterone receptor antagonist, a second amount of an alpha-adrenergic modulating agent, and one or more pharmaceutically acceptable carrier materials, wherein said first amount and said second amount together comprise a therapeutically-effective amount of said aldosterone receptor antagonist and said alpha-adrenergic modulating agent, and wherein said composition exhibits a release profile, determined using a suitable release profile test, in which more than about 20% by weight of the aldosterone receptor antagonist is released from the composition at about four hours after initiation of the test.

51. The composition of claim 50 wherein the release profile test is conducted according to U.S. Pharmacopeia 24, Test No. 711, using apparatus 2 at 50 rpm, with an aqueous dissolution medium containing 1% sodium dodecyl sulfate at 37° C., and wherein release is measured by dissolution of the aldosterone receptor antagonist in the medium.

52. The pharmaceutical composition of claim 51 wherein said alpha-adrenergic modulating agent is an alpha-1-adrenergic antagonist.

53. The pharmaceutical composition of claim 51 wherein said alpha-adrenergic modulating agent is an alpha-2-adrenergic antagonist.

54. The pharmaceutical composition of claim 51 wherein said alpha-adrenergic modulating agent is selected from the group consisting of dapiprazole, doxazosin, labetalol, prazosin, tamsulosin, tolazoline, phenoxybenzamine, phentolamine, terazosin, apraclonidine, clonidine, guanfacine and guanabenz.

55. The pharmaceutical composition of claim 51 wherein said alpha-adrenergic modulating agent is selected from the group consisting of amosulalol, arotinolol, fenspirlde, indoramin, naftopidil, nicergoline, trimazosin, yohimbine, bunazosin, urapidil, alfuzosin, ketanserin, monatepil, SUN 9221, S-2150, guanfacil, rilmenidine, and moxonidine.

56. The pharmaceutical composition of claim 51 wherein said aldosterone receptor antagonist is an epoxy-steroidal aldosterone receptor antagonist.

57. The pharmaceutical composition of claim 56 wherein said epoxy-steroidal aldosterone receptor antagonist has an epoxy moiety fused to the “C” ring of the steroidal nucleus of a 20-spiroxane compound.

58. The pharmaceutical composition of claim 57 wherein said 20-spiroxane compound is characterized by the presence of a 9α-,11α-substituted epoxy moiety.

59. The pharmaceutical composition of claim 56 wherein said epoxy-steroidal aldosterone receptor antagonist is selected from the group consisting of:
Eplerenone;
Pregn-4-ene-7,21-dicarboxylic acid, 9,11-epoxy-17-hydroxy-3-oxo-dimethyl ester, (7α,11α,17β)-;
3′H-cyclopropa[6,7]pregna-4,6-diene-21-carboxylic acid, 9,11-epoxy-6,7-dihydro-17-hydroxy-3-oxo-, γ-lactone, (6β,7β,11α,17β)-;
Pregn-4-ene-7,21-dicarboxylic acid, 9,11-epoxy-17-hydroxy-3-oxo, 7-(1-methylethyl) ester, monopotassium salt, (7α,11α,17β)-;
Pregn-4-ene-7,21-dicarboxylic acid, 9,11-epoxy-17-hydroxy-3-oxo-, 7-methyl ester, monopotassium salt, (7α,11α,17β)-;
3′H-cyclopropa[6,7]pregna-1,4,6-triene-21-carboxylic acid, 9,11-epoxy-6,7-dihydro-17-hydroxy-3-oxo-, γ-lactone, (6β,7β,11α)-;
3′H-cyclopropa[6,7]pregna-4,6-diene-21-carboxylic acid, 9,11-epoxy-6,7-dihydro-17-hydroxy-3-oxo-, methyl ester, (6β,7β,11α,17β)-;
3′H-cyclopropa[6,7]pregna-4,6-diene-21-carboxylic acid, 9,11-epoxy-6,7-dihydro-17-hydroxy-3-oxo-, monopotassium salt, (6β,7β,11α,17β)-;
3′H-cyclopropa[6,7]pregna-4,6-diene-21-carboxylic acid, 9,11-epoxy-6,7-dihydro-17-hydroxy-3-oxo-, γ-lactone, (6β,7β,11α,17β)-;
Pregn-4-ene-7,21-dicarboxylic acid, 9,11-epoxy-17-hydroxy-3-oxo-, γ-lactone, ethyl ester, (7α,11α,17β)-; and
Pregn-4-ene-7,21-dicarboxylic acid, 9,11-epoxy-17-hydroxy-3-oxo-, γ-lactone, 1-methylethyl ester, (7α,11α,17β)-.

60. The pharmaceutical composition of claim 56 wherein said epoxy-steroidal aldosterone receptor antagonist is eplerenone.

61. The pharmaceutical composition of claim 60 wherein said alpha-adrenergic modulating agent is selected from the group consisting of amosulalol, arotinolol, dapiprazole, doxazosin, fenspirlde, indoramin, labetalol, naftopidil, nicergoline, prazosin, tamsulosin, tolazoline, trimazosin, yohimbine, phenoxybenzamine, phentolamine, terazosin, bunazosin, urapidil, alfuzosin, ketanserin, monatepil, SUN 9221, and S-2150.

62. The pharmaceutical composition of claim 61 wherein said alpha-adrenergic modulating agent and said aldosterone receptor antagonist are present in said combination in a weight ratio range from about one-to-one to about one-to-twenty of said alpha-adrenergic modulating agent to said aldosterone receptor antagonist.

63. The pharmaceutical composition of claim 62 wherein said weight ratio range is from about one-to-five to about one-to-fifteen.

64. The pharmaceutical composition of claim 62 wherein said weight ratio is about one-to-ten.

65. The pharmaceutical composition of claim 61 wherein said first amount of eplerenone is between about 0.1 mg to about 400 mg.

66. The pharmaceutical composition of claim 60 wherein said alpha-adrenergic modulating agent is selected from the group consisting of clonidine, apraclonidine, guanfacine, guanabenz, guanfacil, rilmenidine, and moxonidine.

67. The pharmaceutical composition of claim 66 wherein said alpha-adrenergic modulating agent and said aldosterone receptor antagonist are present in said combination in a weight ratio range from about one-to-one to about one-to-twenty of said alpha-adrenergic modulating agent to said aldosterone receptor antagonist.

68. The pharmaceutical composition of claim 67 wherein said weight ratio range is from about one-to-five to about one-to-fifteen.

69. The pharmaceutical composition of claim 67 wherein said weight ratio is about one-to-ten.

70. The pharmaceutical composition of claim 66 wherein said first amount of eplerenone is between about 0.1 mg to about 400 mg.

71. The pharmaceutical composition of claim 51 wherein said aldosterone receptor antagonist is spironolactone.

72. The pharmaceutical composition of claim 71 wherein said alpha-adrenergic modulating agent is selected from the group consisting of amosulalol, arotinolol, dapiprazole, doxazosin, fenspirlde, indoramin, labetalol, naftopidil, nicergoline, prazosin, tamsulosin, tolazoline, trimazosin, yohimbine, phenoxybenzamine, phentolamine, terazosin, bunazosin, urapidil, alfuzosin, ketanserin, monatepil, SUN 9221, and S-2150.

73. The pharmaceutical composition of claim 71 wherein said alpha-adrenergic modulating agent is selected from the group consisting of clonidine, apraclonidine, guanfacine, guanabenz, guanfacil, rilmenidine, and moxonidine.

74. The pharmaceutical composition of claim 71 wherein said alpha-adrenergic modulating agent and said aldosterone receptor antagonist are present in said composition in a weight ratio range from about one-to-one to about one-to-twenty of said alpha-adrenergic modulating agent to said aldosterone receptor antagonist.

75. A method for the treatment or prevention of a cardiovascular disorder in a subject susceptible to or afflicted with such disorder comprising administering to the subject a first amount of an aldosterone receptor antagonist and: a second amount of an alpha-adrenergic modulating agent, wherein said first and second amount together comprise a therapeutically-effective amount of said aldosterone receptor antagonist and said alpha-adrenergic modulating agent, and wherein the aldosterone receptor antagonist is administered to the subject in the form of a composition exhibiting a release profile, determined using a suitable release profile test, in which more than about 20% by weight of the aldosterone receptor antagonist is released from the composition at about four hours after initiation of the test.

76. The method of claim 75 wherein the release profile test is conducted according to U.S. Pharmacopeia 24, Test No. 711, using apparatus 2 at 50 rpm, with an aqueous dissolution medium containing 1% sodium dodecyl sulfate at 37° C., and wherein release is measured by dissolution of the aldosterone receptor antagonist in the medium.

77. The method of claim 76, wherein said cardiovascular disorder is selected from the group consisting of hypertension, heart failure, cirrhosis and ascites.

78. The method of claim 76, wherein said cardiovascular disorder is hypertension.

79. The method of claim 76, wherein said cardiovascular disorder is heart failure.

80. The method of claim 76 wherein said aldosterone receptor antagonist and said alpha-adrenergic modulating agent are administered in a sequential manner.

81. The method of claim 76 wherein said aldosterone receptor antagonist and said alpha-adrenergic modulating agent are administered in a substantially simultaneous manner.

82. The method of claim 76 wherein said composition further comprises said alpha-adrenergic modulating agent.

83. The method of claim 76 wherein said alpha-adrenergic modulating agent is selected from the group consisting of dapiprazole, doxazosin, labetalol, prazosin, tamsulosin, tolazoline, phenoxybenzamine, phentolamine, terazosin, apraclonidine, clonidine, guanfacine and guanabenz.

84. The method of claim 76 wherein said alpha-adrenergic modulating agent is selected from the group consisting of amosulalol, arotinolol, fenspirlde, indoramin, naftopidil, nicergoline, trimazosin, yohimbine, bunazosin, urapidil, alfuzosin, ketanserin, monatepil, SUN 9221, S-2150, guanfacil, rilmenidine, and moxonidine.

85. The method of claim 76 wherein said aldosterone receptor antagonist is an epoxy-steroidal aldosterone receptor antagonist.

86. The method of claim 85 wherein said epoxy-steroidal aldosterone receptor antagonist has an epoxy moiety fused to the “C” ring of the steroidal nucleus of a 20-spiroxane compound.

86. The method of claim 86 wherein said 20-spiroxane compound is characterized by the presence of a 9α-,11α-substituted epoxy moiety.

87. The method of claim 85 wherein said epoxy-steroidal aldosterone receptor antagonist is selected from the group consisting of:
Eplerenone;
Pregn-4-ene-7,21-dicarboxylic acid, 9,11-epoxy-17-hydroxy-3-oxo-dimethyl ester, (7α,11α,17β)-;
3′H-cyclopropa[6,7]pregna-4,6-diene-21-carboxylic acid, 9,11-epoxy-6,7-dihydro-17-hydroxy-3-oxo-, γ-lactone, (6β,7β,11α,17β)-;
Pregn-4-ene-7,21-dicarboxylic acid, 9,11-epoxy-17-hydroxy-3-oxo, 7-(1-methylethyl) ester, monopotassium salt, (7α,11α,17β)-;
Pregn-4-ene-7,21-dicarboxylic acid, 9,11-epoxy-17-hydroxy-3-oxo-, 7-methyl ester, monopotassium salt, (7α,11α,17β)-;
3′H-cyclopropa[6,7]pregna-1,4,6-triene-21-carboxylic acid, 9,11-epoxy-6,7-dihydro-17-hydroxy-3-oxo-, γ-lactone, (6β,7β,11α)-;
3′H-cyclopropa[6,7]pregna-4,6-diene-21-carboxylic acid, 9,11-epoxy-6,7-dihydro-17-hydroxy-3-oxo-, methyl ester, (6β,7β,11α,17β)-;
3′H-cyclopropa[6,7]pregna-4,6-diene-21-carboxylic acid, 9,11-epoxy-6,7-dihydro-17-hydroxy-3-oxo-, monopotassium salt, (6β,7β,11α,17β)-;
3′H-cyclopropa[6,7]pregna-4,6-diene-21-carboxylic acid, 9,11-epoxy-6,7-dihydro-17-hydroxy-3-oxo-, γ-lactone, (6β,7β,11α,17β)-;
Pregn-4-ene-7,21-dicarboxylic acid, 9,11-epoxy-17-hydroxy-3-oxo-, γ-lactone, ethyl ester, (7α,11α,17β)-; and
Pregn-4-ene-7,21-dicarboxylic acid, 9,11-epoxy-17-hydroxy-3-oxo-, γ-lactone, 1-methylethyl ester, (7α,11α,17β)-.

88. The method of claim 85 wherein said epoxy-steroidal aldosterone receptor antagonist is eplerenone.

89. The method of claim 88 wherein said alpha-adrenergic modulating agent is selected from the group consisting of dapiprazole, doxazosin, labetalol, prazosin, tamsulosin, tolazoline, phenoxybenzamine, phentolamine, terazosin, apraclonidine, clonidine, guanfacine and guanabenz.

90. The method of claim 88 wherein said alpha-adrenergic modulating agent is selected from the group consisting of amosulalol, arotinolol, fenspirlde, indoramin, naftopidil, nicergoline, trimazosin, yohimbine, bunazosin, urapidil, alfuzosin, ketanserin, monatepil, SUN 9221, S-2150, guanfacil, rilmenidine, and moxonidine.

91. The method of claim 88 wherein said alpha-adrenergic modulating agent and said aldosterone receptor antagonist are administered in a weight ratio range from about one-to-one to about one-to-twenty of said alpha-adrenergic modulating agent to said aldosterone receptor antagonist.

92. The method of claim 91 said weight ratio range is from about one-to-five to about one-to-fifteen.

93. The method of claim 91 wherein said weight ratio is about one-to-ten.

94. The method of claim 88 wherein said eplerenone is administered in a daily dose range from about 0.1 mg to about 400 mg.

95. The method of claim 88 wherein said eplerenone is administered in a daily dose range from about 1 mg to about 200 mg.

96. The method of claim 88 wherein said eplerenone is administered in a daily dose range from about 10 mg to about 100 mg.

97. The method of claim 88 wherein said eplerenone is administered in a daily dose selected from the group consisting of 25 mg, 50 mg and 100 mg.

98. The method of claim 76 wherein said aldosterone antagonist is spironolactone.

99. The method of claim 98 wherein said alpha-adrenergic modulating agent is selected from the group consisting of dapiprazole, doxazosin, labetalol, prazosin, tamsulosin, tolazoline, phenoxybenzamine, phentolamine, terazosin, apraclonidine, clonidine, guanfacine and guanabenz.

100. The method of claim 98 wherein said alpha-adrenergic modulating agent is selected from the group consisting of amosulalol, arotinolol, fenspirlde, indoramin, naftopidil, nicergoline, trimazosin, yohimbine, bunazosin, urapidil, alfuzosin, ketanserin, monatepil, SUN 9221, S-2150, guanfacil, rilmenidine, and moxonidine.

101. The method of claim 98 wherein said alpha-adrenergic modulating agent and said aldosterone receptor antagonist are present in said combination in a weight ratio range from about one-to-one to about one-to-twenty of said alpha-adrenergic modulating agent to said aldosterone receptor antagonist.

102. A kit for the treatment or prevention of a cardiovascular disorder comprising an aldosterone receptor antagonist and an alpha-adrenergic modulating agent.

103. The kit of claim 102 further comprising written instructions for the use of said kit by a subject.

104. The kit of claim 103 wherein the written instructions state how the subject can use said kit to obtain a therapeutic effect without inducing unwanted side-effects.

105. The kit of claim 103 wherein the written instructions comprise all or a part of the product label approved by a drug regulatory agency for said kit.
